(19) United States
(12) Patent Application Publication
(10) Pub. No.: US 2009/0264420 A1
(43) Pub. Date: Oct. 22, 2009

(54) ORGANIC COMPOUNDS

(76) Inventors: Gary Michael Ksander, Amherst, NH (US); Qi-Ying Hu, Needham, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/438,811

(22) PCT Filed: Aug. 23, 2007
(86) PCT No.: PCT/US07/18660

§ 371 Date: Feb. 25, 2009

Related U.S. Application Data
(60) Provisional application No. 60/823,562, filed on Aug. 25, 2006.

(51) Int. Cl. A61K 31/519; C07D 487/04; A61K 31/551; A61P 9/00; A61P 3/00

(52) U.S. Cl. 514/221; 544/281; 544/231; 540/502; 514/259.5

(57) ABSTRACT

The present invention provides a compound of formula I:

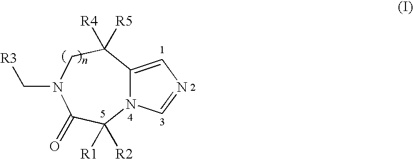

said compound is inhibitor of aldosterone synthase, and/or 11 beta-hydroxylase (CYPL11B1), and/or aromatase, and thus can be employed for the treatment of a disorder or disease mediated by aldosterone synthase, aromatase, or CYPL11B1. Accordingly, the compound of formula I can be used in treatment of hypokalemia, hypertension, congestive heart failure, renal failure, in particular, chronic renal failure, restenosis, atherosclerosis, syndrome X, obesity, nephropathy, post-myocardial infarction, coronary heart diseases, increased formation of collagen, fibrosis and remodeling following hypertension and endothelial dysfunction. Finally, the present invention also provides a pharmaceutical composition.

The present invention relates to novel imidazole derivatives that are used as aldosterone synthase inhibitors, and/or 11 beta-hydroxylase inhibitors (CYP11B1), and/or aromatase inhibitors, as well as for treatment of a disorder or disease mediated by aldosterone synthase, CYP 11 B1, or aromatase.

The present invention provides a compound of formula (I):

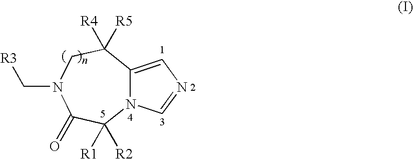

wherein

n is 0 or 1;

R₂ is hydrogen; or

R₁ and R₂ are independently alkyl, non-aromatic heterocyclyl, cycloalkyl, cycloalkyl-alkyl, alkenyl, or alkynyl; or

R₁ and R₂ together with the carbon atom to which they are attached optionally form a 3- to 7-membered ring;

R₃ is heterocyclyl, alkyl, haloalkyl, cycloalkyl, aryl, or heteroaryl, each of which is optionally substituted with one to three substituents selected from alkyl, halogen, trifluoromethyl, cyano, alkoxy, cycloalkyl, hydroxy or cycloalkyl-alkyl;

R₄ and R₅ are independently hydrogen, halogen, hydroxy, or alkyl; or

a pharmaceutically acceptable salt thereof; or an optical isomer thereof; or a mixture of optical isomers.

Preferably, the present invention provides the compound of formula (I), wherein n is 0 or 1; R₂ is hydrogen; or R₁ and R₂, are independently (C₁-C₇) alkyl, (4- to 9-membered)-non-aromatic heterocyclyl, (C₁-C₇) alkenyl, (C₁-C₇) alkynyl, (C₃-C₇) cycloalkyl, or (C₃-C₇) cycloalkyl-(C₁-C₇) alkyl; R₃ is (4- to 9-membered)-non-aromatic heterocyclyl, (C₁-C₇) alkyl, (C₁-C₇) haloalkyl, (C₃-C₇) cycloalkyl, (C₆-C₁₀) aryl or (C₆-C₁₀) heteroaryl, each of which is optionally substituted with one to three substituents selected from (C₁-C₇) alkyl, halogen, trifluoromethyl, cyano, (C₁-C₇) alkoxy, (C₃-C₇) cycloalkyl, or hydroxy; R₄ and R₅ are independently hydrogen, halogen, hydroxy, or (C₁-C₇) alkyl; or R₁ and R₂ together with the carbon atom to which they are attached optionally form a 3- to 7-membered ring; or a pharmaceutically acceptable salt thereof; or an optical isomer thereof; or a mixture of optical isomers.

Also preferably, the present invention provides the compound of formula (I), wherein R₂ is hydrogen; or R₁ and R₂ are independently (C₁-C₇) alkyl, (4- to 7-membered)-non-aromatic heterocyclyl, (C₃-C₇) cycloalkyl or (C₃-C₇) cycloalkyl-(C₁-C₇) alkyl; R₃ is (4- to 7-membered)-heterocyclyl, (C₁-C₇) alkyl, (C₁-C₇) haloalkyl, (C₃-C₇) cycloalkyl, (C₃-C₇) cycloalkyl-(C₁-C₇) alkyl, (C₆-C₁₀) aryl or (C₆-C₁₀) heteroaryl, each of which is optionally substituted with one to three substituents selected from (C₁-C₇) alkyl, halogen, trifluoromethyl, cyano, (C₁-C₇) alkoxy, (C₃-C₇) cycloalkyl, or hydroxy; R₄ and R₅ are independently hydrogen, or (C₁-C₇) alkyl; or R₁ and R₂ together with the carbon atom to which they are attached optionally form a 3- to 7-membered ring; or a pharmaceutically acceptable salt thereof; or an optical isomer thereof; or a mixture of optical isomers.

Also preferably, the present invention provides the compound of formula (I); wherein

n is 0 or 1; R₁ is hydrogen or (C₁-C₇) alkyl; R₂ is (C₃-C₇) cycloalkyl, (C₃-C₇) cycloalkyl-(C₁-C₇) alkyl, or (C₁-C₇) alkenyl; R₃ is (4- to 7-membered)-heterocyclyl, (C₁-C₇) alkyl, (C₃-C₇) cycloalkyl, or (C₆-C₁₀) aryl, each of which is optionally substituted with one to three substituents selected from (C₁-C₇) alkyl, halogen, trifluoromethyl, cyano, (C₁-C₇) alkoxy, or hydroxy; R₄ and R₅ are independently hydrogen; or R₁ and R₂ together with the carbon atom to which they are attached optionally form a 3- to 7-membered ring; or pharmaceutically acceptable salts thereof; or an optical isomer thereof; or a mixture of optical isomers.

Also preferably, the present invention provides the compound of formula (I), wherein n is 0 or 1; R₁ is hydrogen or (C₁-C₇) alkyl; R₂ is (C₁-C₇) alkyl; R₃ is (C₃-C₇) cycloalkyl, or (C₆-C₁₀) aryl, each of which is optionally substituted with one to three substituents selected from (C₁-C₇) alkyl, halogen, trifluoromethyl, cyano, (C₁-C₇) alkoxy, or hydroxy; R₄ and R₅ are independently hydrogen; or R₁ and R₂ together with the carbon atom to which they are attached optionally form a 3- to 7-membered ring; or pharmaceutically acceptable salts thereof; or an optical isomer thereof, or a mixture of optical isomers.

For purposes of interpreting this specification, the following definitions will apply and whenever appropriate, terms used in the singular will also include the plural and vice versa.

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 7 carbon atoms, or 1 to 4 carbon atoms. Representative examples of alkyl include, but are not limited to; methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein, the term “haloalkyl” refers to an alkyl as defined herein, that is substituted by one or more halo groups as defined herein. Preferably the haloalkyl can be monohaloalkyl, dihaloalkyl or polyhaloalkyl including perhaloalkyl. A monohaloalkyl can have one iodo, bromo, chloro or fluoro within the alkyl group. Dihaloalky and polyhaloalkyl groups can have two or more of the same halo atoms or a combination of different halo groups within the alkyl. Preferably; the polyhaloalkyl contains up to 12, or 10, or 8, or 6; or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

The term “aryl” refers to monocyclic or bicyclic aromatic hydrocarbon groups having 6-20 carbon atoms in the ring portion. Preferably, the aryl is a (C₆-C₁₀) aryl. Non-limiting examples include phenyl, biphenyl, naphthyl or tetrahydronaphthyl, each of which may optionally be substituted by 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—aryl-S—, nitro, cyano, carboxy, alkyl-O—C(O)—, carbamoyl, alkyl-S(O)—, sulfonyl, sulfonamido, heterocyclyl and the like.

Furthermore, the term “aryl” as used herein, refers to an aromatic substituent which can be a single aromatic ring, or multiple aromatic rings that are fused together, linked covalently, or linked to a common group such as a methylene or ethylene moiety. The common linking group also can be a carbonyl as in benzophenone or oxygen as in diphenylether or nitrogen as in diphenylamine.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Preferably, alkoxy groups have about 1-7, more preferably about 1-4 carbons.

As used herein, the term “acyl” refers to a group R—C(O)— of from 1 to 10 carbon atoms of a straight, branched, or cyclic configuration or a combination thereof, attached to the parent structure through carbonyl functionality. Such group can be saturated or unsaturated, and aliphatic or aromatic. Preferably, R in the acyl residue is alkyl, or alkoxy, or aryl, or heteroaryl. Also preferably, one or more carbons in the acyl residue may be replaced by nitrogen, oxygen or sulfur as long as the point of attachment to the parent remains at the carbonyl. Examples of acyl include but are not limited to, acetyl, benzoyl, propionyl, isobutyryl, t-butoxycarbonyl, benzyloxycarbonyl and the like. Lower acyl refers to acyl containing one to four carbons,

As used herein, the term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aryl-alkyl-NHC(O)—, alkyl(aryl-alkyl)-NC(O)— and the like.

As used herein, the term “sulfonyl” refers to R—SO₂—, wherein R is hydrogen, alkyl, aryl, hereoaryl, aryl-alkyl, heteroaryl-alkyl, alkoxy, aryloxy, cycloalkyl, or heterocyclyl.

As used herein, the term “sulfonamido” refers to alkyl-S(O)₂—NH—, aryl-S(O)₂—NH—, aryl-alkyl-S(O)₂—NH—, heteroaryl-S(O)₂—NH—, heteroaryl-alkyl-S(O)₂—NH—, alkyl-S(O)₂—N(alkyl)-, aryl-S(O)₂—N(alkyl)-, aryl-alkyl-S(O)₂—N(alkyl)-, heteroaryl-S(O)₂—N(alkyl)-, heteroaryl-alkyl-S(O)₂—N(alkyl)- and the like.

As used herein, the term “heterocyclyl” or “heterocyclo” refers to an optionally substituted, fully saturated or unsaturated, aromatic or nonaromatic cyclic group, e.g., which is a 4- to 7-membered monocyclic, 7- to 12-membered bicyclic or 10- to 15-membered tricyclic ring system, which has carbon atoms and at least one heteroatom in at least one carbon atom-containing ring. Each ring of the heterocyclic group containing a heteroatom can have 1, or 2 or 3 heteroatoms selected from nitrogen atoms, oxygen atoms and sulfur atoms, where the nitrogen and sulfur heteroatoms can also optionally be oxidized to various oxidation states. The heterocyclic group can be attached at a heteroatom or a carbon atom. The heterocyclyl can include fused or bridged rings as well as spirocyclic rings.

Exemplary monocyclic heterocyclic groups include pyrrolidinyl, pyrrolyl, pyrazolyl, oxetanyl, pyrazolinyl, imidazolyl, imidazolinyl, imidazolidinyl, triazolyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl, sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl; furopyridinyl (such as furo[2,3-c]pyridinyl, furo[3,2-b]-pyridinyl) or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” further refers to heterocyclic groups as defined herein substituted with 1, 2 or 3 substituents selected from the groups consisting of the following:

(a) alkyl;

(b) hydroxy (or protected hydroxy);

(c) halo;

(d) oxo, i.e., ═O;

(e) amino, alkylamino or dialkylamino;

(f) alkoxy;

(g) cycloalkyl;

(h) carboxyl;

(i) heterocyclooxy, wherein heterocyclooxy denotes a heterocyclic group bonded through an oxygen bridge;

(j) alkyl-O—C(O)—;

(k) mercapto;

(l) nitro;

(m) cyano;

(n) sulfamoyl or sulfonamido;

(O) aryl;

(p) alkyl-C(O)—O—;

(q) aryl-C(O)—O—;

(r) aryl-S—;

(s) aryloxy;

(t) alkyl-S—;

(u) formyl, i.e., HC(O)—;

(v) carbamoyl;

(w) aryl-alkyl-; and

(x) aryl substituted with alkyl, cycloalkyl, alkoxy, hydroxy, amino, alkyl-C(O)—NH—, alkylamino, dialkylamino or halogen.

As used herein, the term “cycloalkyl” refers to saturated or unsaturated monocyclic, bicyclic or tricyclic hydrocarbon groups of 3-12 carbon atoms, preferably 3-9, or 3-7 carbon atoms, each of which can be optionally substituted by one, or two, or three, or more substituents, such as alkyl, halo, oxo, hydroxy, alkoxy, alkyl-C(O)—, acylamino, carbamoyl, alkyl-NH—, (alkyl)₂N—, thiol, alkyl-S—, nitro, cyano, carboxy, alkyl-O—C(O)—, sulfonyl, sulfonamido, sulfamoyl, heterocyclyl and the like. Exemplary monocyclic hydrocarbon groups include, but are not limited to, cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]-hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

As used herein, the term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, (aryl-alkyl)-NHS(O)₂—, (heteroaryl-alkyl)-NHS(O)₂— and the like.

As used herein, the term “aryloxy” refers to both an —O-aryl and an —O-heteroaryl group, wherein aryl and heteroaryl are defined herein.

As used herein, the term “heteroaryl” refers to a 5-14 membered monocyclic- or bicyclic- or polycyclic-aromatic ring system, having 1 to 8 heteroatoms selected from N, O or S: Preferably, the heteroaryl is a 5-10 or 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl.

The term “heteroaryl” also refers to a group in which a heteroaromatic ring is fused to one or more aryl, cycloaliphatic, or heterocyclyl rings, where the radical or point of attachment is on the heteroaromatic ring. Nonlimiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinoliyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-; 4-, 5-, 6-, 7-, or 8-4aH carbazolyl, 1-, 2-, 3-, 4-, 5-, 6-, 7-, or 8-carbzaolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-; 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-; 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6″-; 7-, 8-; or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2′,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-; 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroary groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

A heteroaryl group may be mono-, bi-, tri-, or polycyclic, preferably mono-, bi-, or tricyclic, more preferably mono- or bicyclic.

As used herein, the term “halogen” or “halo” refers to fluoro, chloro, bromo, and iodo.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system: When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)- or (S)—. The present invention is meant to include all such possible isomers, including racemic mixtures, optically pure forms and intermediate mixtures. Optically active (R)- and (S)— isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable salts” refers to salts that retain the biological effectiveness and properties of the compounds of this invention and, which are not biologically or otherwise undesirable. In many bases, the compounds of the present invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed, with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate-acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton; Pa., (1985), which is herein incorporated by reference.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by aldosterone synthase or aromatase, or (ii) associated with aldosterone synthase activity or aromatase activity, or (iii) characterized by abnormal activity of aldosterone synthase or aromatase; or (2) reducing or inhibiting the activity of aldosterone synthase or aromatase; or <3) reducing or inhibiting the expression of aldosterone synthase or aromatase. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of aldosterone synthase or aromatase; or at least partially reducing or inhibiting the expression of aldosterone synthase or aromatase.

As used herein, the term “subject” refers to an animal. Preferably, the animal is a mammal. A subject also refers to for example, primates (e.g., humans), cows, sheep, goats, horses, dogs, cats, rabbits, rats, mice, fish, birds and the like. In a preferred embodiment, the subject is a human.

As used herein, the term “a disorder” or “a disease” refers to any derangement or abnormality of function; a morbid physical or mental state. See Dordand's Illustrated Medical Dictionary, (W.B. Saunders Co. 27th ed. 1988).

As used herein, the term “inhibition” or “inhibiting” refers to the reduction or suppression of a given condition, symptom, or disorder, or disease, or a significant decrease in the baseline activity of a biological activity or process. Preferably, the condition or symptom or disorder or disease is mediated by aldosterone synthase activity or aromatase. More preferably, the condition or symptom or disorder or disease is associated with the abnormal activity of aldosterone synthase or aromatase, or the condition or symptom or disorder or disease is associated with the abnormal expression of aldosterone synthase or aromatase.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least, one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “abnormal” refers to an activity or feature which differs from a normal activity or feature.

As used herein, the term “abnormal activity” refers to an activity which differs from the activity of the wild-type or native gene or protein, or which differs from the activity of the gene or protein in a healthy subject. The abnormal activity can be stronger or weaker than the normal activity. In one embodiment, the “abnormal activity” includes the abnormal (either over- or under-) production of mRNA transcribed from a gene. In another embodiment, the “abnormal activity” includes the abnormal (either over- or under-) production of polypeptide from a gene. In another embodiment, the abnormal activity refers to a level of a mRNA or polypeptide that is different from a normal level of said mRNA or polypeptide by about 15%, about 25%, about 35%, about 50%, about 65%, about 85%, about 100% or greater. Preferably, the abnormal level of the mRNA or polypeptide can be either higher or lower than the normal level of said mRNA or polypeptide. Yet in another embodiment, the abnormal activity refers to functional activity of a protein that is different from a normal activity of the wild-type protein. Preferably, the abnormal activity can be stronger or weaker than the normal activity. Preferably, the abnormal activity is due to the mutations in the corresponding gene, and the mutations can be in the coding region of the gene or non-coding regions such as transcriptional promoter regions. The mutations can be substitutions, deletions, insertions.

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Any asymmetric carbon atom on the compounds of the present invention can be present in the (R) —, (S)— or (R,S)— configuration, preferably in the (R)- or (S)— configuration. Substituents at atoms with unsaturated bonds may, if possible, be present in cis-(Z)- or trans-(E)-form. Therefore, the compounds of the present invention can be in the form of one of the possible isomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, the imidazolyl moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Finally, compounds of the present invention are either obtained in the free form, as a salt thereof, or as prodrug derivatives thereof.

When a basic group is present in the compounds of the present invention, the compounds can be converted into acid addition salts thereof, in particular, acid addition salts with the imidazolyl moiety of the structure, preferably pharmaceutically acceptable salts thereof. These are formed, with inorganic acids or organic acids. Suitable inorganic acids include but are not limited to, hydrochloric acid, sulfuric acid, a phosphoric or hydrohalic acid. Suitable organic acids include but are not limited to, carboxylic acids, such as (C₁-C₄)alkanecarboxylic acids which, for example, are unsubstituted or substituted by halogen, e.g., acetic acid, such as saturated or unsaturated dicarboxylic acids, e.g., oxalic, succinic, maleic or fumaric acid, such as hydroxycarboxylic acids, e.g., glycolic, lactic, malic, tartaric or citric acid, such as amino acids, e.g., aspartic or glutamic acid, organic sulfonic acids, such as (C₁-C₄)alkylsulfonic acids, e.g., methanesulfonic acid; or arylsulfonic acids which are unsubstituted or substituted, e.g., by halogen. Preferred are salts formed with hydrochloric acid, methanesulfonic acid and maleic acid.

When an acidic group is present in the compounds of the present invention, the compounds can be converted into salts with pharmaceutically acceptable bases. Such salts include alkali metal salts, like sodium, lithium and potassium salts; alkaline earth metal salts, like calcium and magnesium salts; ammonium salts with organic bases, e.g., trimethylamine salts, diethylamine salts, tris(hydroxymethyl)methylamine salts, dicyclohexylamine salts and N-methyl-D-glucamine salts; salts with amino acids like arginine, lysine and the like. Salts may be formed using conventional methods, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

When both a basic group and an acid group are present in the same molecule, the compounds of the present invention can also form internal salts.

The present invention also provides pro-drugs of the compounds of the present invention that converts in vivo to the compounds of the present invention. A pro-drug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds are inactive or have low activity compared to the corresponding active drug compound, that contains one or more protective groups and are converted to an active form by metabolism or solvolysis. Both the active drug form and any released metabolic products should have acceptably low toxicity. Typically, the formation of active drug compound involves a metabolic process or reaction that is one of the follow types:

1. Oxidative reactions, such as oxidation of alcohol, carbonyl, and acid functions, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-delakylatiori, oxidative O- and S-delakylation, oxidative deamination, as well as other oxidative reactions.

2. Reductive reactions, such as reduction of carbonyl groups, reduction of alcoholic groups and carbon-carbon double bonds, reduction of nitrogen-containing functions groups, and other reduction reactions.

3. Reactions without change in the state of oxidation, such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain-polymers or other moieties, such as cyclodextrins. See, Cheng et al., US20040077595, application Ser. No. 10/656,838, incorporated herein by reference. Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, The Practice of Medicinal Chemistry, Ch. 31-32, Ed. Werriuth, Academic Press, San Diego, Calif., 2001.

Exemplary prodrugs are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters; such as the pivaloyloxymethyl ester and the like conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

In view of the close relationship between the compounds, the compounds in the form of their salts and the pro-drugs, any reference to the compounds of the present invention is to be understood as referring also to the corresponding pro-drugs of the compounds of the present invention, as appropriate and expedient.

Furthermore, the compounds of the present invention, including their salts, can also be obtained in the form of their hydrates, or include other solvents used for their crystallization.

The compounds of the present invention have valuable pharmacological properties. The compounds of the present invention are useful as aldosterone synthase inhibitors. Aldosterone synthase is a mitcohcondrial cytochrome P450 enzyme catalyzing the last step of aldosterone production in the adrenal cortex, i.e., the conversion of 11-deoxycorticosterone to aldosterone. Aldosterone synthase has been demonstrated to be expressed in all cardiovascular tissues such as heart, umbilical cord, mesenteric and pulmonary arteries, aorta, endothelium and vascular cells. Moreover, the expression of aldosterone synthase is closely correlated with aldosterone production in cells. It has been observed that elevations of aldosterone activity induces different diseases such as congestive heart failure, myocardial fibrosis, ventricular arrhythmia and other adverse effects, etc. Accordingly, the compounds of the present invention as aldosterone synthase inhibitors, are also useful for treatment of a disorder or disease characterized by abnormal activity of aldosterone synthase. Preferably, the compounds of the present invention are also useful for treatment of a disorder or disease selected from hypokalemia, hypertension, congestive heart failure, renal failure, in particular, chronic renal failure, restenosis, atherosclerosis, syndrome X, obesity, nephropathy, post-myocardial infarction, coronary heart diseases, increased formation of collagen, fibrosis and remodeling following hypertension and endothelial dysfunction.

Furthermore, the compounds of the present inventions are useful as aromatase inhibitors. Aromatase is a cytochrome P450 enzyme, it plays a central role in the extragonadal biosynthesis of estrogens such as estradiol, estrone and estrol, and is widely distributed in muscular and adipose tissue (Longcope C, Pratt J H. Schneider S H, Fineberg S E, 1977, J. Clin. Endocrinol. Metab. 45:1134-1145). An increase in aromatase activity has been confirmed to be associated with estrogen-dependent disorders or diseases. Accordingly, the compounds of the present invention are also useful for treatment of a disorder or disease characterized by abnormal expression of aromatase. Preferably, the compounds of the present invention are useful for treatment of an estrogen-dependent disorder or disease. More preferably, the compounds of the present invention are useful for treatment of an estrogen-dependent disorder or disease selected from gynecomastia, osteoporosis, prostate cancer, endometriosis, uterine fibroids, dysfunctional uterine bleeding, endometrial hyperplasia, polycystic ovarian disease, infertility, fibrocystic breast disease, breast cancer and fibrocystic mastopathy.

Furthermore, the compounds of the present invention are useful as CYP11B1 (11-β-hydroxylase) inhibitors. CYP11B1 catalyzes the last steps of cortisol synthesis. Cortisol is the main glucocorticoid in human. It regulates energy mobilization and thus the stress response. In addition, it is involved in the immune response of the human body. Abnormally increased cortisol level is the cause of a variety of diseases including Cushing's syndrome. Accordingly, the compounds of the present invention as CYP11 B1 inhibitors are also useful for the treatment of a disorder or a disease or a condition characterized by abnormal activity or abnormal level of CYP11B1. The compounds of the present invention can be used for the treatment of a disorder, a disease or a condition such as Cushing's syndrome, excessive CYP11B1 level, the ectopic ACTH syndrome, the change in adrenocortical mass, primary pigmented nodular adrenocortical disease (PPNAD) Carney complex (CNC), anorexia nervosa, chronic alcoholic poisoning, nicotine or cocaine withdrawal syndrome, the post-traumatic stress syndrome, the cognitive impairment after a stroke and the cortisol-induced mineralocorticoid excess, etc.

Additionally, the present invention provides:
a compound of the present invention for use as a medicament;
the use of a compound of the present invention for the preparation of a pharmaceutical composition for the delay of progression and/or treatment of a disorder or disease mediated by aldosterone synthase, or characterized by abnormal activity of aldosterone synthase, or by abnormal expression of aldosterone synthase.

the use of a compound of the present invention for the preparation of a pharmaceutical composition for the delay of progression and/or treatment of a disorder or disease selected from hypokalemia, hypertension, congestive heart failure, renal failure, in particular, chronic renal failure, restenosis, atherosclerosis, syndrome X, obesity, nephropathy, post-myocardial infarction, coronary heart diseases, increased formation of collagen, fibrosis and remodeling following hypertension and endothelial dysfunction.

Additionally, the present invention provides:
a compound of the present invention for use as a medicament;
the use of a compound of the present invention for the preparation of a pharmaceutical composition for the delay of progression and/or treatment of a disorder or disease mediated by aromatase, or responsive to inhibition of aromatase, or characterized by abnormal activity or expression of aromatase.
the use of a compound of the present invention for the preparation of a pharmaceutical composition for the delay of progression and/or treatment of a disorder or disease selected from gynecomastia, osteoporosis, prostate cancer, endometriosis, uterine fibroids, dysfunctional uterine bleeding, endometrial hyperplasia, polycystic ovarian disease, infertility, fibrocystic breast disease, breast cancer and fibrocystic mastopathy.

Additionally, the present invention provides:
a compound of the present invention for use as a medicament;
the use of a compound of the present invention for the preparation of a pharmaceutical composition for the delay of progression and/or treatment of a disorder or disease or condition mediated by CYP 11 B1, or characterized by abnormal activity of CYP 11 B1, or by abnormal expression/level of CYP11B1.

the use of a compound of the present invention for the preparation of a pharmaceutical composition for the delay of progression and/or treatment of a disorder or disease or condition selected from Cushing's syndrome, excessive CYP 11 B1 level, the ectopic ACTH syndrome, the change in adrenocortical mass, primary pigmented nodular adrenocortical disease (PPNAD) Carney complex (CNC), anorexia nervosa, chronic alcoholic poisoning, nicotine or cocaine withdrawal syndrome, the post-traumatic stress syndrome, the cognitive impairment after a stroke and the cortisol-induced mineralocorticoid excess, etc.

The compounds of formula (I) can be prepared by the procedures described in the following sections.

Generally, the compounds of formula (I) can be prepared according to Scheme 1. The first part of the synthesis is the preparation of the common intermediate amine 5 by two approaches. In the first method, amine reacts with either commercially available aldehyde 1 (Cas #3301647-6) or ketone 2, which can be prepared by a Grignard addition of 1 followed by Magnesium (IV) dioxide oxidation, to give imine which subsequently undergoes a reduction to yield amine 5. Alternatively, the known alcohol 3 (Cas #127607-62-9, J. Med. Chem. 1996, 39 (19), 3806.) first reacts with methanesulfonic acid chloride and subsequently with amine 4 to give 5. The amine 5 reacts with α-bromo acid chloride 6 to give amide intermediate, which undergoes cyclization after heating and yields (I) as 7. After deprotonation of 7 with LiHMDS followed by alkylation with iodo compound, the di-substituted (I) is yielded. Alternatively, the compounds of formula (I) can also be prepared according to the methods described in WO2004/014914.

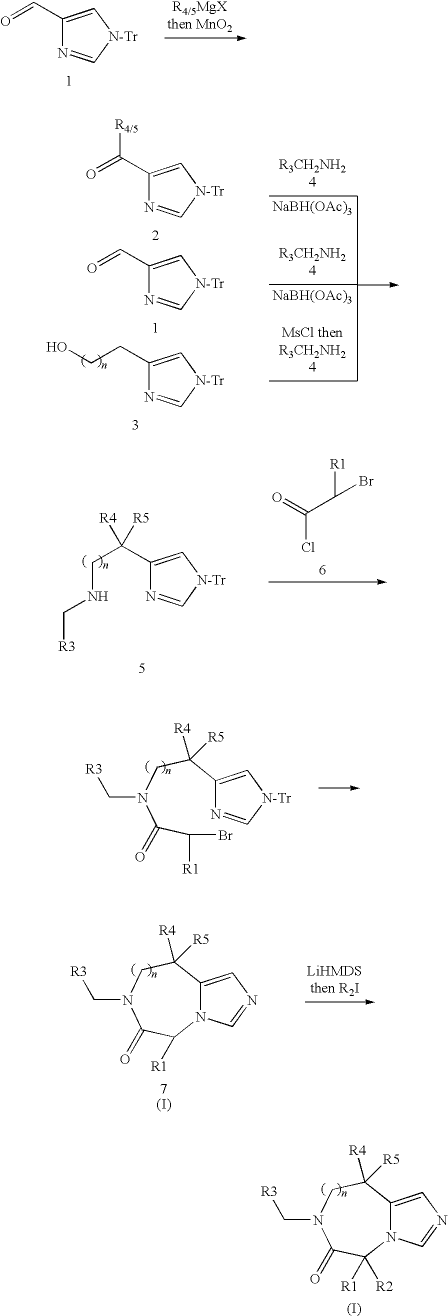

Generally, enantiomers of the compounds of the present invention can be prepared by methods known to those skilled in the art to resolve racemic mixtures, such as by formation and recrystallization of diastereomeric salts or by chiral chromotagraphy or HPLC separation utilizing chiral stationery phases.

In starting compounds and intermediates which are converted to the compounds of the invention in a manner described herein, functional groups present, such as amino, thiol, carboxyl and hydroxy groups; are optionally protected by conventional protecting groups that are common in preparative organic chemistry. Protected amino, thiol, carboxyl and hydroxyl groups are those that can be converted under mild conditions into free amino thiol, carboxyl and hydroxyl groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxyl group, amino group, etc.), the structure and stability of the molecule of which the substituent is a part and the reaction conditions.

Well-known protecting groups that meet these conditions and their introduction and removal are described, e.g., in McOmie, “Protective Groups in Organic Chemistry”, Plenum Press, London, N.Y. (1973); and Greene and Wuts, “Protective Groups in Organic Synthesis”, John Wiley and Sons, Inc., NY (1999).

The above-mentioned reactions are carried out according to standard methods, in the presence or absence of diluent, preferably, such as are inert to the reagents and are solvents thereof, of catalysts, condensing or said other agents, respectively and/or inert atmospheres, at low temperatures, room temperature or elevated temperatures, preferably at or near the boiling point of the solvents used, and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers etc.

Preferably, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable compositions for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

The present invention further provides anhydrous pharmaceutical compositions and dosage forms comprising the compounds of the present invention as active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel-Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The pharmaceutical compositions contain a therapeutically effective amount of a compound of the invention as defined above, either alone or in a combination with one or more therapeutic agents, e.g., each at an effective therapeutic dose as reported in the art. Such therapeutic agents include at least one or two or more selected from the following groups:

(i) angiotensin II receptor antagonist or a pharmaceutically acceptable salt thereof,

(ii) HMG-Co-A reductase inhibitor or a pharmaceutically acceptable salt thereof,

(iii) angiotensin converting enzyme (ACE) Inhibitor or a pharmaceutically acceptable salt thereof,

(iv) calcium channel blocker (CCB) or a pharmaceutically acceptable salt thereof,

(v) dual angiotensin converting enzyme/neutral endopeptidase (ACE/NEP) inhibitor or a pharmaceutically acceptable salt thereof,

(vi) endothelin antagonist or a pharmaceutically acceptable salt thereof,

(vii) renin inhibitor or a pharmaceutically acceptable salt thereof,

(viii) diuretic or a pharmaceutically acceptable salt thereof,

(ix) an ApoA-I mimic;

(x) an anti-diabetic agent;

(xi) an obesity-reducing agent;

(xii) an aldosterone receptor blocker;

(xiii) an endothelin receptor blocker;

(xiv) a CETP inhibitor;

(xv) an inhibitor of Na—K-ATPase membrane pump;

(xvi) a beta-adrenergic receptor blocker or an alpha-adrenergic receptor blocker;

(xvii) a neutral endopeptidase (NEP) inhibitor; and

(xviii) an inotropic agent.

An angiotensin II receptor antagonist or a pharmaceutically acceptable salt thereof is understood to be an active ingredients which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing, structural features, essentially preferred are the non-peptidic ones. For example, mention may be made of the compounds which are selected from the group consisting of valsartan, losartan, candesartan, eprosartan, irbesartan, saprisartan, tasosartan, telmisartan, the compound with the designation E-1477 of the following formula

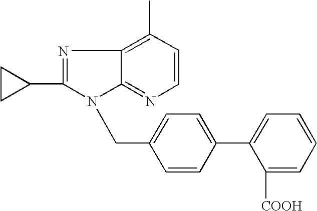

the compound with the designation SC-52458 of the following formula

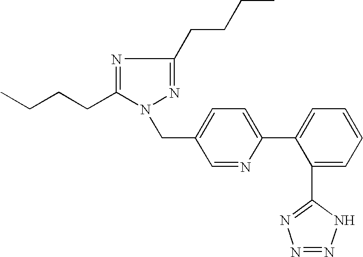

and the compound with the designation ZD-8731 of the following formula

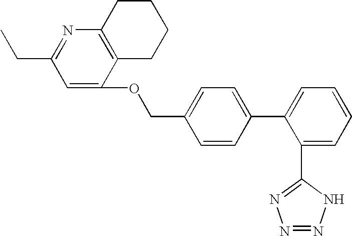

or, in each case, a pharmaceutically acceptable salt thereof.

Preferred AT₁-receptor antagonist are those agents which have been marketed, most preferred is valsartan or a pharmaceutically acceptable salt thereof.

HMG-Co-A reductase inhibitors (also called beta-hydroxy-beta-methylglutaryl-co-enzyme-A reductase inhibitors) are understood to be those active agents that may be used to lower the lipid levels including cholesterol in blood.

The class of HMG-Co-A reductase inhibitors comprises compounds having differing structural features. For example, mention may be made of the compounds that are selected from the group consisting of atorvastatin; cerivastatin, compactin, dalvastatin, dihydrocompactin, fluindbstatin, fluvastatin, lovastatin, pitavastatin, mevastatin, pravastatin, rivastatin, simvastatin, and velostatin, or, in each case, a pharmaceutically acceptable salt thereof.

Preferred HMG-Co-A reductase inhibitors are those agents which have been marketed, most preferred is fluvastatin and pitavastatin or; in each case, a pharmaceutically acceptable salt thereof.

The interruption of the enzymatic degradation of angiotensin I to angiotensin II with so-called ACE-inhibitors (also called angiotensin converting enzyme inhibitors) is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

The class of CCBs essentially comprises dihydropyridines (DHPs) and non-DHPs such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamill, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

A preferred dual angiotensin converting enzyme/neutral endopetidase (ACE/NEP) inhibitor is, for example, omapatrilate (cf. EP 629627), fasidotril or fasidotrilate, or, if appropriable, a pharmaceutically acceptable salt thereof.

A preferred endothelin antagonist is, for example, bosentan (cf. EP 526708 A), furthermore, tezosentan (cf. WO 96/19459), or in each case, a pharmaceutically acceptable salt thereof.

Suitable renin inhibitors include compounds having different structural features. For example, mention may be made of compounds which are selected from the group consisting of ditekiren (chemical name: [1S-[1R*,2R*4R*(1R*,2R*)]]-1-[(1,1-dimethylethoxy)carbonyl]-L-prolyl-L-phenylalanyl-N-[2-hydroxy-5-methyl-1-(2-methylpropyl)-4-[[[2-methyl-1-[[(2-pyridinylmrthyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); and zankiren (chemical name: [1S-[1R*[R*(R*)],2S*,3R*)]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), preferably, in each case, the hydrochloride salt thereof, SPP630, SPP635 and SPP800 as developed by Speedel.

Preferred renin inhibitor of the present invention include RO 66-1132 and RO 66-1168 of formula (A) and (B)

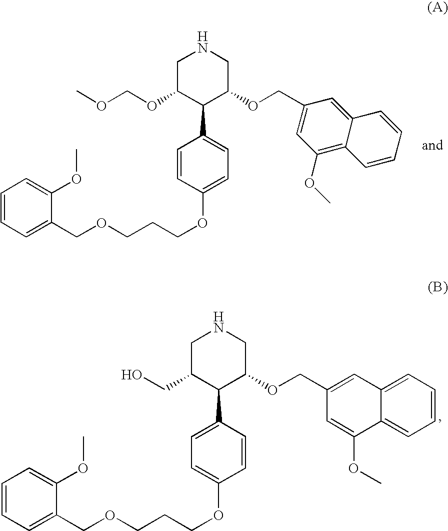

respectively, or a pharmaceutically acceptable salt thereof.

In particular, the present invention relates to a renin inhibitor which is a δ-amino-γ-hydroxy-ω-aryl-alkanoic acid amide derivative of the formula (C)

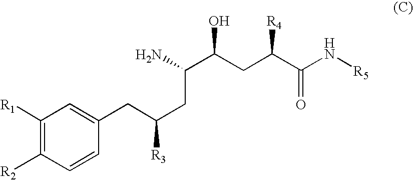

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆-alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁-alkyl, C₁₋₆-aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆-alkyl, C₁₋₆alkyl-HN—C(O)—C₁₋₆-alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4-C atoms. Examples are methoxymethyl, 2-methoxyethyl, 3-methoxypropyl, 4-methoxybutyl, 5-methoxypentyl, 6-methoxyhexyl, ethoxymethyl, 2-ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆-alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (C) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆-alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆-aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxycarbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are 8-amino-γ-hydroxy-ω-aryl-alkanoic acid amide derivatives of formula (C) having the formula

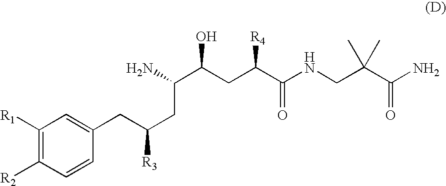

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren.

The term “aliskiren”, if not defined specifically, is to be understood both as the free base and as a salt thereof, especially a pharmaceutically acceptable salt thereof, most preferably a hemi-fumarate salt thereof.

A diuretic is, for example, a thiazide derivative selected from the group consisting of chlorothiazide, hydrochlorothiazide, methylclothiazide, and chlorothalidon. The most preferred is hydrochlorothiazide.

An ApoA-I mimic is, for example, D4F peptide, especially of formula D-W—F—K-A-F—Y-D-K—V-A-E-K—F—K-E-A-F

An anti-diabetic agents include insulin secretion enhancers which are active ingredients that have the property to promote the secretion of insulin from pancreatic β-cells. Examples of insulin secretion enhancers are a biguanide derivative, for example, metformin or, if appropriate, a pharmaceutically acceptable salt thereof, especially the hydrochloride thereof. Other insulin secretion enhancers include sulfonylureas (SU), especially those which promote the secretion of insulin from pancreatic β-cells by transmitting signals of insulin secretion via SU receptors in the cell membrane, including (but are not limited to) tolbutamide; chlorpropamide; tolazamide; acetohexamide; 4-chloro-N-[(1-pyrrolidinylamino)carbonyl]-benzensulfonamide (glycopyramide); glibenclamide (glyburide); gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; and, tolylcyclamide, or pharmaceutically acceptable salts thereof.

Insulin secretion enhancers furthermore include short-acting insulin secretion enhancers, such as the phenylalanine derivative nateglinide [N-(trans-4-isopropylcyclohexyl-carbonyl)-D-phenylalanine] (cf. EP 196222 and EP 526171) of the formula

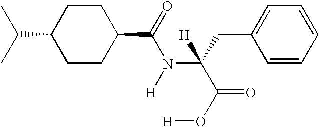

and repaglinide [(S)-2-ethoxy-4-{2-[[3-methyl-1-[2-(1-piperidinyl)phenyl]butyl]amino]-2-oxoethyl}benzoic acid]. Repaglinide is disclosed in EP 589874, EP 147850 A2, in particular Example 11 on page 61, and EP 207331 A1. It can be administered in the form as it is marketed, e.g. under the trademark NovoNorm™; calcium (2S)-2-benzyl-3-(cis-hexahydro-2-isoindolinlycarbonyl)-propionate dihydrate (mitiglinide—cf. EP 507534); furthermore representatives of the new generation of SUs such as glimepiride (cf. EP 31058); in free or pharmaceutically acceptable salt form. The term nateglinide likewise comprises crystal modifications such as disclosed in EP 0526171 B1 or U.S. Pat. No. 5,488,510; respectively, the subject matter of which, especially with respect to the identification, manufacture and characterization of crystal modifications, is herewith incorporated by reference to this application, especially the subject matter of claims 8 to 10 of said U.S. patent (referring to H-form crystal modification) as well as the corresponding references to the B-type crystal modification in EP 196222 B1 the subject matter of which, especially with respect to the identification, manufacture and characterization of the B-form crystal modification. Preferably, in the present invention, the B- or H-type, more preferably the H-type, is used. Nateglinide can be administered in the form as it is marketed e.g. under the trademark STARLIX™.

Insulin secretion enhancers likewise include the long-acting insulin secretion enhancer DPP-IV inhibitors, GLP-1 and GLP-1 agonists.

DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

The DPP-IV inhibitor can be peptidic or, preferably, non-peptidic. DPP-IV inhibitors are in each case generically and specifically disclosed e.g. in WO 98119998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications. Preferred are those compounds that are specifically disclosed in Example 3 of WO 98/19998 and Example 1 of WO 00/34241, respectively.

GLP-1 is a insulinotropic proteine which was described, e.g., by W. E. Schmidt et al. in Diabetologia, 28, 1985; 704-707 and in U.S. Pat. No. 5,705,483.

The term “GLP-1 agonists” used herein means variants and analogs of GLP-1 (7-36)NH₂ which are disclosed in particular in U.S. Pat. No. 5,120,712, U.S. Pat. No. 5,118,666, U.S. Pat. No. 5,512,549, WO 91/11457 and by C. Orskov et al in J. Biol. Chem. 264 (1989) 12826. The term “GLP-1 agonists” comprises especially compounds like GLP-1 (7-37), in which compound, the carboxy-terminal amide functionality of Arg³⁶ is displaced with Gly at the 37^{th} position of the GLP-1(7-36)NH₂ molecule and variants and analogs thereof including GLN⁹-GLP-1 (7-37), D-GLN⁹-GLP-1 (7-37), acetyl LYS⁹-GLP-1 (7-37), LYS¹⁸-GLP-1 (7-37) and, in particular, GLP-1 (7-37)OH, VAL⁸-GLP-1 (7-37), GLY⁸-GLP-1 (7-37), THR⁸-GLP-1 (7-37), MET-GLP-1 (7-37) and 4-imidazopropionyl-GLP-1. Special preference is also given to the GLP agonist analog exendin-4, described by Greig et al in Diabetologia 1999, 42, 45-50.

An insulin sensitivity enhancer restores impaired insulin receptor function to reduce insulin resistance and consequently enhance the insulin sensitivity.

An appropriate insulin sensitivity enhancer is, for example, an appropriate hypoglycemic thiazolidinedione derivative (glitazone).

An appropriate glitazone is, for example, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]-methyl}-thiazolidine-2,4-dione (darglitazone), 5-{[4-(1-methyl-cyclohexyl)methoxy)-phenyl]methyl}thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl-2-hydroxyethoxy]benzyl}thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl])-5-phenysulfonyl)thiazolidine-2,4-dione, 5-[3-(4-chlorophenyl])-2-propynyl])-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297). Preferred are pioglitazone, rosiglitazone and troglitazone.

Other anti-diabetic agents include, insulin signalling pathway modulators, like inhibitors of protein tyrosine phosphatases (PTPases), antidiabetic non-small molecule mimetic compounds and inhibitors of glutamine-fructose-6-phosphate amidotransferase (GFAT); compounds influencing a dysregulated hepatic glucose production, like inhibitors of glucose-6-phosphatase (G6 Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK); pyruvate dehydrogenase kinase (PDHK) inhibitors; inhibitors of gastric emptying; insulin; inhibitors of GSK-3; retinoid X receptor. (RXR) agonists; agonists of Beta-3 AR; agonists of uncoupling proteins (UCPs); non-glitazone type PPARγ agonists; dual PPARα/PPARγ agonists; antidiabetic vanadium containing compounds; incretin hormones, like glucagon-like peptide-1 (GLP-1) and GLP-1 agonists; beta-cell imidazoline receptor antagonists; miglitol; and α₂-adrenergic antagonists; in which the active ingredients are present in each case in free form or in the form of a pharmaceutically acceptable salt.

An obesity-reducing agent includes lipase inhibitors such as orlistat and appetite suppressants such as sibutramine, phentermine.

An aldosteron receptor blocker includes spironolactone and eplerenone.

An endothelin receptor blocker includes bosentan, etc.

A CETP inhibitor refers to a compound that inhibits the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). The CETP inhibitors include those disclosed in U.S. Pat. No. 6,140,343 and U.S. Pat. No. 6,197,786. CETP inhibitors disclosed in these patents include compounds such as [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, which is also known as torcetrapib. CETP inhibitors are also described in U.S. Pat. No. 6,723,752, which includes a number of CETP inhibitors including (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol. CETP inhibitors also include those described in U.S. patent application Ser. No. 10/807,838 filed Mar. 23, 2004. U.S. Pat. No. 5,512,548 discloses certain polypeptide derivatives having activity as CETP inhibitors, also certain CETP-inhibitory rosenonolactone derivatives and phosphate-containing analogs of cholesteryl ester are disclosed in J. Antibiot., 49(8): 815-816 (1996), and Bioorg. Med. Chem. Lett., 6:1951-1954 (1996), respectively. Furthermore, the CETP inhibitors also include those disclosed in WO2000/017165, WO2005/095409 and WO2005/097806.

A Na_K-ATPase inhibitor can be used to inhibit the Na and K exchange across the cell membranes. Such inhibitor can be for example digoxin.

A beta-adrenergic receptor blocker includes but is not limited to: esmolol especially the hydrochloride thereof; acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in, German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41, 491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,7691 nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol; which may be prepared as disclosed in U.S. Pat. No. 3,432,545; and xibenolol, which may be prepared as disclosed in U.S. Pat. No. 4,018,824.

An alpha-adrenergic receptor blocker includes but is not limited to: amosulalbl, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art.

The natriuretic peptides constitute a family of peptides that include the atrial (ANP), brain-derived (BNP) and C-type natriuretic (CNP) peptides. The natriuretic peptides effect vasodilation, natriuresis, diuresis, decreased aldosterone release, decreased cell growth, and inhibition of the sympathetic nervous system and the renin angiotensin-aldosterone system indicating their involvement in the regulation of blood pressure and of sodium and water balance. Neutral endopeptidase 24. 11 (NEP) inhibitors impede degradation of natriuretic peptides and elicit pharmacological actions potentially beneficial in the management of several cardiovascular disorders. A NEP inhibitor useful in the said combination is an agent selected from the group represented by candoxatril, sinorphan, SCH 34826 and SCH 42495.

An inotropic agent is selected from the group consisting of: digoxin, digitoxin, digitalis, dobutamine, dopamine, epinephrine, milrinone, amrinone and norepinephrine, etc.

A compound of the present invention may be administered either simultaneously; before or after the other active ingredient, either separately by the same or different route of administration or together in the same pharmaceutical formulation.

Furthermore, the combinations as described above can be administered to a subject via simultaneous, separate or sequential administration (use). Simultaneous administration (use) can take place in the form of one fixed combination with two or three or more active ingredients, or by simultaneously administering two or three or more compounds that are formulated independently. Sequential administration (use) preferably means administration of one (or more) compounds or active ingredients of a combination at one time point, other compounds or active ingredients at a different time point, that is, in a chronically staggered manner, preferably such that the combination shows more efficiency than the single compounds administered independently (especially showing synergism). Separate administration (use) preferably means administration of the compounds or active ingredients of the combination independently of each other at different time points, preferably meaning that two, or three or more compounds are administered such that no overlap of measurable blood levels of both compounds are present in an overlapping manner (at the same time).

Also combinations of two or three or more of sequential, separate and simultaneous administrations are possible, preferably such that the combination compound-drugs show a joint therapeutic effect that exceeds the effect found when the combination compound-drugs are used independently at time intervals so large that no mutual effect on their therapeutic efficiency can be found, a synergistic effect being especially preferred.

Alternatively, the pharmaceutical compositions contain a therapeutically effective amount of a compound of the invention as defined above, either alone or in a combination with one or more therapeutic agents, e.g., each at an effective therapeutic dose as reported in the art, selected from the group consisting of an antiestrogen; an anti-androgen; a gonadorelin agonist; a topoisomerase I inhibitor; a topoisomerase II inhibitor; a microtubule active agent; an alkylating agent; an anti-neoplastic anti-metabolite; a platin compound; a compound targeting/decreasing a protein or lipid kinase activity or a protein or lipid phosphatase activity, a anti-angiogenic compound; a compound which induces cell differentiation processes; monoclonal antibodies; a cyclooxygenase inhibitor; a bisphosphonate; a heparanase inhibitor; a biological response modifier; an inhibitor of Ras oncogenic isoforms; a telomerase inhibitor; a protease inhibitor, a matrix metalloproteinase inhibitor, a methionine aminopeptidase inhibitor; a proteasome inhibitor; agents which target, decrease or inhibit the activity of Flt-3; an HSP90 inhibitor; antiproliferative antibodies; an HDAC inhibitor; a compound which targets, decreases or inhibits the activity/function of serine/theronine mTOR kinase; a somatostatin receptor antagonist; an anti-leukemic compound; tumor cell damaging approaches; an EDG binder; a ribonucleotide reductase inhibitor; an S-adenosylmethionine decarboxylase inhibitor; a monoclonal antibody of VEGF or VEGFR; photodynamic therapy; an Angiostatic steroid; an implant containing corticosteroids; an AT1 receptor antagonist; and an ACE inhibitor.

Additionally, the present invention provides:
a pharmaceutical composition or combination of the present invention for use as a medicament;
the use of a pharmaceutical composition or combination of the present invention for the delay of progression and/or treatment of a disorder or disease mediated by or associated with aldosterone synthase, or responsive to inhibition of aldosterone synthase, or characterized by abnormal activity or expression of aldosterone synthase.

the use of a pharmaceutical composition or combination of the present invention for the delay of progression and/or treatment of a disorder or disease mediated by or associated with aromatase, or responsive to inhibition of aromatase, or characterized by abnormal activity or expression of aromatase.

the use of a pharmaceutical composition or combination of the present invention for the delay of progression and/or treatment of a disorder or disease selected from hypokalemia, hypertension, congestive heart failure, atrial fibrillation, renal failure, in particular, chronic renal failure, restenosis, atherosclerosis, syndrome X, obesity, nephropathy, post-myocardial infarction, coronary heart diseases, increased formation of collagen, fibrosis such as cardiac or myocardiac fibrosis and remodeling following hypertension and endothelial dysfunction.

the use of a pharmaceutical composition or combination of the present invention for the delay of progression and/or treatment of a disorder or disease selected from gynecomastia, osteoporosis, prostate cancer, endometriosis, uterine fibroids, dysfunctional uterine bleeding, endometrial hyperplasia, polycystic ovarian disease, infertility, fibrocystic breast disease, breast cancer and fibrocystic mastopathy.

The pharmaceutical composition or combination of the present invention can be in unit dosage of about 1-1000 mg of active ingredients for a subject of about 50-70 kg, preferably about 5-500 mg or about 10-250 mg or about 10-150 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously; e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, preferably between about 1-100mg/kg.

The activities of a compound according to the present invention can be assessed by the following in vitro & in vivo methods well-described in the art. See Fieber, A et al. (2005), “Aldosterone Synthase Inhibitor Ameliorates Angiotensin II-induced Organ Damage,” Circulation, 111:3087-3094. The reference cited herein is incorporated by reference in its entirety.

In particular, the aldosterone, synthase and aromatase inhibitory activities in vitro can be determined by the following assays.

Human adrenocortical carcinoma NCI-H295R cell line is obtained from American Type Culture Collection (Manassas, Va.). Insulin/transferrin/selenium (ITS)-A supplement (100×), DMEM/F-12, antibiotic/antimycotic (100×), and fetal calf serum (FCS) are purchased from Gibco (Grand Island, N.Y.). Anti-mouse PVT scintillation proximity assay (SPA) beads and NBS 96-well plates are obtained from Amersham (Piscataway, N.J.) and Corning (Acton, Mass.), respectively. Solid black 96-well flat bottom plates are purchased from Costar (Corning, N.Y.). Aldosterone and angiotensin (Ang II) are purchased from Sigma (St. Louis, Mo.). D-[1,2,6,7⁻³H(N)]aldosterone was acquired from PerkinElmer (Boston, Mass.). Nu-serum was a product of BD Biosciences (Franklin Lakes, N.J.). The NADPH regenerating system, dibenzylfluorescein (DBF), and human aromatase Supersomes® are obtained from Gentest (Woburn, Mass.).

For in vitro measurement of aldosterone activity, human adrenocortical carcinoma NCI-H295R cells are seeded in NBS 96-well plates at a density of 25,000 cells/well in 100 μl′ of a growth medium containing DMEM/F12 supplemented with 10% FCS, 2.5% Nu-serum, 1 μg ITS/ml, and 1× antibiotic/antimycotic. The medium is changed after culturing for 3 days at 37° C. under an atmosphere of 5% CO₂/95% air. On the following day, cells are rinsed with 100 μl of DMEM/F12 and incubated with 100 μl of treatment medium containing 1 μM Ang II and a compound at different concentrations in quadruplicate wells at 37° C. for 24 hr. At the end of incubation, 50 μl of medium is withdrawn from each well for measurement of aldosterone production by an RIA using mouse anti-aldosterone monoclonal antibodies.

Measurement of aldosterone activity can also be performed using, a 96-well plate format. Each test sample is incubated with 0.02 μCi of D-[1,2,6,7-³H(N)]aldosterone and 0.3 μg of anti-aldosterone antibody in phosphate-buffered saline (PBS) containing 0.1% Triton X-100, 0.1% bovine serum albumin, and 12% glycerol in a total volume of 200 μl at room temperature for 1 hr. Anti-mouse PVT SPA beads (50 μl) are then added to each well and incubated overnight at room temperature prior to counting in a Microbeta plate counter. The amount of aldosterone in each sample is calculated by comparing with a standard curve generated using known quantities of the hormone.

To measure aromatase activity, the human aromatase assay is performed in 96-well flat bottom plates according to a published protocol (Stresser et al, 2000) with minor modifications. Briefly, 10 μl, of an NADPH regenerating system containing 2.6 mM NADP⁺, 6.6 mM glucose 6-phosphate, 6.6 mM MgCl₂, and 0.8 U/ml glucose-6-phosphate dehydrogenase in 50 mM potassium phosphate, pH 7.4, is pre-incubated with the test compound at a desired concentration at 30° C. for 10 min in a total volume of 100 μl. Afterwards, 4 pmol of human aromatase, 20 μg of control microsomal protein, and 4 μM DBF in 100 μl of 50 mM potassium phosphate, pH 7.4, is added to each well and incubated at 30° C. for 90 min. The reaction is terminated by the addition of 75 μl of 2 N NaOH to each well. After 2 hr, the product, fluorescein, is measured by a fluorimeter using excitation and emission wavelengths of 485 and 538 nm, respectively.

Full concentration-response curves of the test compound are performed at least 3 times. The IC₅₀ values are derived using a non-linear least squares curve-fitting program from Microsoft XLfit.

The in vivo inhibitory activities for aldosterone synthase and aromatase can be determined by the following assays.

Test compounds (i.e., potential aldosterone synthase inhibitors) are profiled in vivo in a conscious rat model of acute secondary hyperaldosteronism. Wild-type rats are instrumented with chronically indwelling arterial and venous cannulas, which are exteriorized through a tether/swivel system. The ambulatory rats are housed in specialized cages to allow blood sampling and parenteral drug administration without disturbing the animals. Angiotensin II is continuously infused intravenously at a level sufficient to elevate plasma aldosterone concentration (PAC) by ˜200-fold to 1-5 nM. This PAC increase is sustained at a stable level for at least 8-9 hours. Test compounds are administered p.o. (via oral gavage) or parenterally (via the arterial catheter) after one hour of angiotensin II infusion at a time when PAC has increased to a steady-state level. Arterial blood samples are collected before and at various times (up to 24 hours) after test agent administration for later determination of PAC and concentration of test agent. From these measurements, various parameters can be derived, e.g., 1) onset and duration of PAC reduction by the test agent, 2) pharmacokinetic parameters of the test agent such as half-life, clearance, volume of distribution, and oral biovailability, 3) dose/PAC response, dose/test-agent concentration, and test-agent concentration/PAC response relationships, and 4) dose- and concentration-potencies and efficacy of the test agent. A successful test compound decreases PAC in a dose- and time-dependent fashion in the dose range of about 0.01 to about 10 mg/kg i.a. or p.o.

The in vitro inhibitory activities for CYP 11 B1 can be determined by the following assay.

The cell line NCI-H295R was originally isolated from an adrenocortical carcinoma and has been characterized in the literature through the stimulable secretion of steroid hormones and the presence of the enymes essential for steroidogenesis. Thus, the NCI-H295R cells have Cyp 11 B1 (steroid 11 p-hydroxylase). The cells show the physiological property of zonally undifferentiated human foetal adrenocortical cells which, however, have the capacity to produce the steroid hormones which are formed in the three, phenotypically distinguishable zones in the adult adrenal, cortex.

The NCI-H295R cells (American Type Culture Collection, ATCC, Rockville, Md., USA) are grown in Dulbeoco's Modified Eagle Ham F-12 Medium (DME/F12), which has been I supplemented with Ulroser SF Serum (Soprachem, Cergy-Saint-Christophe, France), insulin, transferrin, selenite (1-T-S, Becton Dickinson Biosiences, Franklin lakes, NJ, USA) and antibiotics in 75 cm² cell culture vessels at 37° C. and in a 95% air-5% carbon dioxide atmosphere. The cells are subsequently transferred for colony formation into a 24-well incubation vessel. They are cultivated there in DME/F12 medium, which is now supplemented with 0.1% bovine serum instead of Ultroser SF for 24 hours. The experiment is initiated by cultivating the cells in DME/F12 medium which is supplemented with 0.1% bovine serum albumin and test compound, in the presence or absence of cell stimulants, for 72 hours. The test substance is added in a concentration range from 0.2 nanomolar to 20 millimolar. Cell stimulants which can be used are angiotensin II (1 D or 100 nanomolar), potassium ions (16 millimolar), forskolin (10 micromolar) or a combination of two stimulants.

The excretion of aldosterone, cortisol, corticosterone and estradiol/estrone into the culture medium can be detected and quantified by commercially available; specific monoclonal antibodies in radioimmunoassays in accordance with the manufacturer's instructions.

Inhibition of the release of certain steroids can be used as a measure of the respective enzyme inhibition by the added test compounds. The dose-dependent inhibition of enzymic activity by a compound is calculated by means of an inhibition plot which is characterized by an IC50.

The IC50 values for active test compounds are ascertained by a simple linear regression analysis in order to construct inhibition plots without data weighting. The inhibition plot is calculated by fting a 4-parameter logistic function to the raw data points using the least squares method. The equation of the 4-parameter logistic function is calculated as follows: Y=(d−a)/((1+(x/c)b))+aI where: a=minimum data level b gradient I c=ICED d=maximum data level x=inhibitor concentration.

TABLE 1
Inhibitory Activity of Compounds

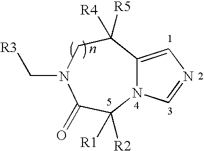

| | | | | | | | AS | 11B1 | ARO |
|---|---|---|---|---|---|---|---|---|---|
| | | | | | R4, | | IC₅₀ | % I @ | % I @ |
| | | R1 | R2 | R3 | R5 | n | (nM) | 100 nM | 10 μM |
| 1 | ent-2 | t-butyl | ethyl | 4-F-Phenyl | H, H | 0 | 139 | | 18 |
| 2 | Ent-2 | 1,1-di-methyl- | H | 3-F-Phenyl | H, H | 1 | 48 | 100 | 60 |
| | | propyl | | | | | | | |
| 3 | Ent-1 | i-propyl | H | cyclohexyl | H, H | 0 | 11 | | 48 |
| 4 | | (CH₂)₅ | | 4-F-Phenyl | H, H | 0 | 73 | | 99 |
| 5 | Ent-1 | cyclobutyl | H | 4-F-Phenyl | H, H | 0 | 18 | | 93 |
| 6 | Ent-2 | t-butyl | H | 4-Cl-Phenyl | H, H | 0 | 3 | 98 | 51 |
| 7 | Ent-1 | 1,1-di-methyl- | H | cyclohexyl | H, H | 0 | 7 | | 50 |
| | | propyl | | | | | | | |
| 8 | Ent-2 | cyclopropyl | H | 4-CN- | H, H | 0 | 66 | 95 | 74 |
| | | | | Phenyl | | | | | |
| 9 | Ent-1 | i-propyl | H | 3-F-Phenyl | H, H | 0 | 231 | | 94 |
| 10 | Ent-1 | 1,1-di-methyl- | H | 4- | H, H | 0 | 190 | | |
| | | propyl | | tetrahydro- | | | | | |
| | | | | pyranyl | | | | | |
| 11 | Ent-1 | cyclobutyl | H | 4-F-Phenyl | H, H | 1 | 197 | | 99 |

Ent-1: the first eluting enantiomer.
Ent-2: the second eluting enantiomer.
AS: aldosterone synthase;
ARO: Aromatase;
11B1: CYP11B1;
I % percentage of inhibitory rate.

### Abbreviations

DCM: dichloromethane
DIBAL: diisobutylaluminum hydride

### DMAP: N,N-dimethylaminopyridine

DME: dimethoxyethane

### DMF: N, N-dimethylformamide

DMSO: dimethylsulfoxide
ESI: electrospray ionization
h: hours
HPLC: high pressure liquid chromatography
HRMS: high resolution mass spectrometry
IPA/i-PrOH: isopropyl alcohol
IR: infrared spectroscopy
LAH: lithium aluminum hydride
LCMS: liquid chromatography/mass spectrometry
LDA: lithium diisoproylamide
LHMDS/LiHMDS: lithium hexamethyldisilazide
min: minutes
MS: mass spectrometry

### NBS: N-bromosuccinimide

NMR: nuclear magnetic resonance
TBSCI: tert-butyldimethylsilyl chloride
TFA: trifluoroacetic acid
THF: tetrahydrofuran
TMEDA: tetramethylethylenediamine
TBS: tert-butyl dimethylsilyl
TMSCl: trimethylsilyl chloride
TLC: thin layer chromatography
Tr: trityl
tᵣ: retention time

### EXAMPLES

The following examples are intended to illustrate the invention and are not to be construed as being limitations thereon. Temperatures are given in degrees centrigrade. If not mentioned otherwise, all evaporations are performed under reduced pressure, preferably between about 15 mm Hg and 100 mm Hg (=20-133 nm bar). The structure of final products, intermediates and starting materials is confirmed by standard analytical methods, e.g., microanalysis and spectroscopic characteristics, e.g., MS, IR, NMR. Abbreviations used are those conventional in the art. The compounds in the following examples have been found to have IC₅₀ values in the range of about 0.1 nM to about 100,00.00 nM for aldosterone synthase.

### Example 1

### A. [5-(tert-Butyl-dimethyl-silanyloxymethyl)-imidazol-1-yl]-acetic acid methyl ester

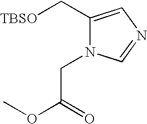

Bromo-acetic acid methyl ester (3.3 g, 21.6 mmol) is added to a solution of 4-(tert-Butyl-dimethyl-silanyloxymethyl)-1-trityl-1H-imidazole (9.1 g, 20 mmol) in acetonitrile (200 mL) at room temperature. After refluxing for 4 h, the resulting mixture is concentrated, and the residue is dissolved into MeOH (200 mL). The resulting mixture is refluxed for 2 h. After concentration, the residue is dissolved into CH₂Cl₂ (150 mL). The solution is washed with water, NaHCO₃ (sat.), brine, and dried over anhydrous Na₂SO₄. After filtration and concentration, the residue is purified by silica gel chromatography and yielded the title compound and immediately used ‘as is’ in the next step.

### B. (5-Hydroxymethyl-imidazol-1-yl)-acetic acid methyl ester

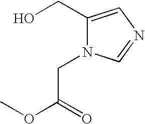

A solution of HCl in ether (1 M, 10 mL, 10 mmol) is added to a solution of [5-(tert-Butyl-dimethyl-silanyloxymethyl)-imidazol-1-yl]-acetic acid methyl ester (0.75 g, 2.6 mmol) in MeOH (10 mL) at room temperature. After stirring for 2 h at room temperature, the resulting solution is diluted with CH₂Cl₂ (40 mL) and saturated NaHCO₃ solution (10 mL). The organic layer is separated and the aqueous layer was extracted with CH₂Cl₂ (30 mL×3). The combined organic layer is washed with saturated NaHCO₃ solution, brine and dried over anhydrous Na₂SO₄. After concentration, the residue is purified by flash column and yielded the title product (385 mg). MS (ESI) m/z 171.2 (M+H).

### C. (5-Formyl-imidazol-1-yl)-acetic acid methyl ester

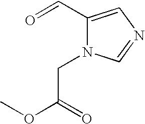

MnO₂ (2.8 g, 27 mmol) is added to a solution of (5-Hydroxymethyl-imidazol-1-yl)-acetic acid methyl ester (0.385 g, 2.26 mmol) in 1,4-dioxane (20 mL, dry) at room temperature. The resulting mixture is refluxed for 4 h, and then cooled to room temperature. After filtration and concentration, the residue is filtered through a pad of silica gel and gave the title compound (273 mg). MS (ESI) m/z 168.3 (M+H).

### D. 7-(4-Fluoro-benzyl)-7,8-dihydro-imidazo[1,5-a]pyrazin-6-one

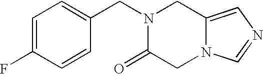

4-F-Benzylamine (0.811 mL, 6.9 mmol) is added to a solution of (5-Formyl-imidazol-1-yl)-acetic acid methyl ester (0.97 g, 5.8 mmol) in 1,2-dichloroethane (35 mL) at 0° C. After 10 min at this temperature, Na(OAc)₃BH (3.86 g, 17.3 mmol) is added. The resulting mixture is stirred for overnight at 23° C. NaHCO₃ (sat.) is poured into the reaction mixture. The organic layer is separated, and the aqueous phase is extracted with CH₂Cl₂ (25 mL×4). The combined extracts are washed with brine, and dried over anhydrous Na₂SO₄. After filtration and concentration, the residue is purified by silica gel chromatography and gave 4-[7-(4-Chloro-benzyl)-6-oxo-5,6,7,8-tetrahydro-imidazo[1,5-a]pyrazin-5-yl]-3-methoxy-benzonitrile (0.92 g, 65% yield). MS (ESI) m/z 246.2 (M+H). ¹H NMR (400 MHz, CDCl₃) δ 7.41 (s, 1H), 7.25-7.16 (m, 2H), 6.94-6.90 (m, 2H), 6.76 (s, 1H), 4.65 (s, 2H), 4.60 (s, 2H), 4.36 (s, 2H).

### E. S-Ethyl-7-(4-fluoro-benzyl)-7,8-dihydro-imidazo[1,5-a]pyrazin 6-one

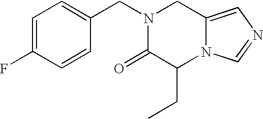

A solution of LiHMDS (0.183 mL, 1 M in THF) is added dropwise to a stirred solution of 7-(4-Fluoro-benzyl)-7,8-dihydro-imidazo[1,5-a]pyrazin-6-one (30 mg, 0.122 mmol) in anhydrous THF (3 mL) at −78° C. After 1 h at this temperature, Etl (14 □l; 0.135 mmol) was added. The resulting mixture is stirred for 5 h at −78° C. Saturated NH₄Cl water solution is added, and extracted with CH₂Cl₂ (10 mL×3). The combined extracts are washed with brine and dried over anhydrous Na₂SO₄. After filtration and concentration, the crude product is purified by silica gel chromatography and give the title compound (7.6 mg). MS (ESI) m/z 274.2 (M+H). ¹H NMR (400 MHz, CDCl₃) δ 7.50 (s, 1H), 7.21-7.18 (m, 21), 6.99-6.94 (m, 2H), 6.82 (s, 1H), 4.735 (d, J=12 Hz, 1H), 4.565 (d, J=12 Hz, 1H), 4.35 (s, 2H), 2.07-2.00 (m, 2H), 0.81 (t, J=8 Hz, 6H).

The following compounds are synthesized analogously.

### 7-(4-Fluoro-benzyl)-5-propyl-7,8-dihydro-imidazo[1,5-a]pyrazin-6-one

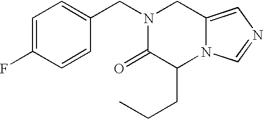

MS (ESI) m/z 288.3 (M+H). ¹H NMR (400 MHz, CDCl₃) δ 7.40 (s, 1H), 7.19-7.16 (m, 2H), 6.96-6.91 (m, 2H), 6.76 (s, 1H), 4.73 (t, J=8.0 Hz, 1H), 4.64 (d, J=16 Hz, 1H), 4.605 (d, J=16 Hz, 1H), 4.33 (s, 2H), 1.90 (q, J=8.0 Hz, 2H), 1.26-1.16 (m, 2H), 0.83 (t, J=8.0 Hz, 3H).

### 5-Butyl-7-(4-fluoro-benzyl)-7,8-dihydro-imidazo[1,5-a]pyrazin-6-one

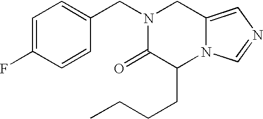

MS (ESI) m/z 302.3 (M+H). ¹H NMR (400 MHz, CDCl₃) δ 7.48 (s, 1H), 7.29-7.23 (m, 2H), 7.06-7.00 (m, 2H), 6.86 (s, 1H), 4.83-4.80, (m, 1H), 4.735 (d, J=16 Hz, 1H), 4.66 (d, J=16 Hz, 1H), 4.40 (s, 2H), 2.05-1.99 (m, 2H), 1.34-1.16 (m, 4H), 0.86 (t, J=8.0 Hz, 3H).

### Example 2

### 5,5-Diethyl-7-(4-fluoro-benzyl)-7,8-dihydro-imidazo[5-a]pyrazin-6-one

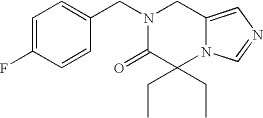

A solution of LiHMDS (0.428 mL, 1 M in THF) is added dropwise to a stirred solution of 7-(4-Fluoro-benzyl)-7,8-dihydro-imidazo[1,5-a]pyrazin-6-one (35 mg, 0.142 mmol) in anhydrous THF (4 mL) at −78° C. After 1 h at this temperature, Etl (38 □l, 0.357 mmol) is added. The resulting mixture is stirred for 4 h at −78° C., then slowly warmed to room temperature. Saturated NH₄Cl solution is added, and extracted with CH₂Cl₂ (20 mL×3). The combined extracts are washed with brine and dried over anhydrous Na₂SO₄. After filtration and concentration, the crude product is purified by silica gel chromatography and give the title compound (23 mg). MS (ESI) m/z 302.2 (M+H). ¹H NMR (400 MHz, CDCl₃) δ 7.38 (s, 1H), 7.21-7.16 (m, 2H), 6.92-6.88 (m, 2H), 6.73 (s, 1H), 4.61 (s, 2H), 4.35 (s, 2H), 2.33-2.24 (m, 2H), 1.85-1.76 (m, 2H), 0.51 (t, J=8 Hz, 6H).

The following compounds are synthesized analogously by employing I (CH₂)ₙI (n 4 or 5) as reagents instead of Etl.

### 7′-(4-fluorobenzyl)-7′,8′-dihydro-6′H-spiro[cyclopentane-1,5′-imidazole[1,5-a]pyrazin]-6′-one

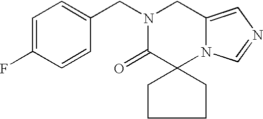

MS (ESI) m/z 300.3 (M+H). ¹H NMR (400 MHz, CDCl₃) δ 7.53 (s, 1H), 7.27-7.23 (m, 2H), 7.05-6.99 (m, 2H), 6.84 (s, 1H), 4.69 (s, 2H), 4.42 (s, 2H), 2.64-2.57 (m, 2H); 2.08-1.91 (m, 6H).

### 7′-(4-fluorobenzyl)-7′,8′-dihydro-6′H-spiro[cyclohexane-1,5′-imidazole[1,5-a]pyrazin]-6-one

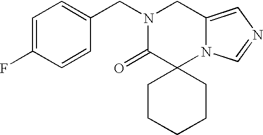

MS (ESI) m/z 314.3 (M+H). ¹H NMR (400 MHz, CDCl₃) δ 7.79 (s, 1H), 7.25-7.18 (m, 2H), 7.05-7.00 (m, 2H), 6.86 (s, 1H), 4.66 (s, 2H), 4.38 (s, 2H), 2.35-2.30 (m, 2H), 2.02-1.88 (m, 4H), 1.73-1.56 (m, 4H).

### Example 3

### 5-tert-Butyl-7-(4-fluoro-benzyl)-7,8-dihydro-imidazo[1,5-a]pyrazin-6-one

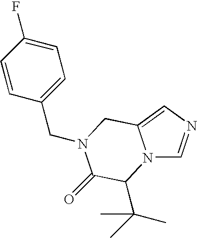

### A. (4-Fluoro-benzyl)-(1-trityl-1H-imidazol-4-ylmethyl)-amine

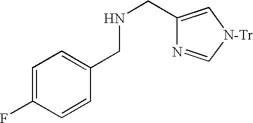

Na(OAc)₃BH (9.39 g, 44.3 mmol) is added slowly to a solution of 1-Trityl-1H-imidazole-4-carbaldehyde (5 g, 14.8 mmol) and 4-Fluoro-benzylamine (2.09 mL, 17.7 mmol) in 300 mL of dry CH₂Cl₂ at room temperature. The resulting mixture is stirred for overnight. The reaction is quenched by 50 mL of saturated NaHCO₃ solution. The organic layer is separated and aqueous layer is extracted with CH₂Cl₂ (50 mL×4). The combined organic layer is washed with brine, dried over anhydrous Na₂SO₄. After filtration and concentration, the residue is purified by flash column and give a yellow solid (4.72 g, 71% yield).

All other amine derivatives can be prepared with the similar method from commercially available amines.

### B. 2-Bromo-3,3-dimethyl-butyryl chloride

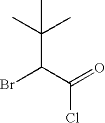

A mixture of 3,3-Dimethyl-butyric acid (8 g, 68.9 mmol), SOCl₂ (32.8 g, 20 mL, 275.6 mmol) and CCl₄ (dry, 8 mL) is heated to 65° C. After 45 min, the resulting mixture is cooled to room temperature, NBS (14.7 g, 82.7 mmol), CCl₄ (dry, 20 mL) and concentrated HBr (48% in water, 7 drops) are added successively. The mixture is heated to 70° C. for 10 min then slowly warmed to 80° C. After 1.5 h, the mixture is cooled back to room temperature, and the solvent is removed under reduced pressure. The solid is separated by filtration and washed with CCl₄. The combined solution is concentrated and the residue is purified by distillation under vacuo, and a slightly yellow oil (6.8 g) is yielded.

The following bromides can be prepared with similar method from the corresponding carboxylic acids.

### 2-Bromo-3-cyclopropyl-3-methyl-butyryl chloride

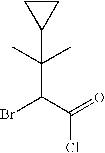

### 2-Bromo-3,3-dimethyl-pentanoyl chloride

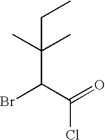

### Bromo-cyclopropyl-acetyl chloride

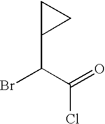

### Bromo-cyclobutyl-acetyl chloride

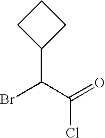

### Bromo-cyclopentyl-acetyl chloride

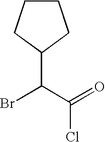

### Bromo-cyclohexyl-acetyl chloride

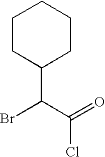

### Bromo-(tetrahydro-pyran-4-yl)-acetyl chloride

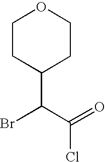

### C. 2-Bromo-N-(4-fluoro-benzyl)-3,3-dimethyl-N-(1-trityl-1H-imidazol-4-ylmethyl)-butyramide

2-Bromo-3,3-dimethyl-butyryl chloride (1.23 g, 5.82 mmol) is added dropwise to a solution of (4-fluoro-benzyl)-(1 trityl-1H-imidazol-4-ylmethyl)-amine (2.17 g, 4.85 mmol) and Et₃N (1.8 mL, 12.1 mmol) in CH₂Cl₂ (30 mL) at 0° C. After 3 h at this temperature, the mixture is slowly warmed up to room temperature and stirred for overnight. The solvent is evaporated and saturated NaHCO₃ solution is added. The aqueous layer is extracted with CH₂Cl₂ (30 mL×4), and the combined organic layer is washed with brine and dried over anhydrous Na₂SO₄. After filtration and concentration, a residue is obtained and used in the next step without further purification.

### D. 5-tert-Butyl-7-(4-fluoro-benzyl)-7,8-dihydro-imidazo[1,5-a]pyrazin-6-one

A solution of 2-bromo-N-(4-fluoro-benzyl)-3,3-dimethyl-N-(1-trityl-1h-imidazol-4-ylmethyl)-butyramide (1.9 b, 3.0 mmol) in Acetonitrile (20 mL) is heated at 13° C. by microwave for 6 h. after concentration, 30 mL of methanol is added and the mixture is heated to reflux for 1.5 h. The solvent is removed, saturated NaHCO₃ solution is added. The mixture is extracted with CH₂Cl₂ (60 mL×3). The combined extracts are washed with brine, and dried over anhydrous Na₂SO₄. After filtration and concentration, the residue is purified by flash column and give 497 mg of title compound. MS (ESI) m/z 302.1 (M+H). ¹H NMR (400 MHz, CDCl₃) δ ppm 1.04 (s, 9H), 4.21-4.45 (m, 2H), 4.49 (s, 11H), 4.56 (d, J=14.40 Hz, 1H), 4.82 (d, J=14.40 Hz, 1H), 6.89 (s, 1H), 6.97-7.10 (m, 2H) 7.22-7.34 (m, 2H), 7.48 (s, 1H). Resolution of the enantiomers is achieved by chiral HPLC using the ChiralPak IA column with a 3% Ethanol in Acetonitrile as mobile phase to give enantiomers with retention time tᵣ=11.8 min and tᵣ=13.2 min.

The following compounds can be prepared with similar method.

### 7-(4-Fluoro-benzyl)-5-isopropyl-7,8-dihydro-imidazo[1,5-a]pyrazin-6-one

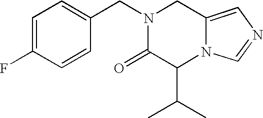

MS (ESI) m/z 288.3 (M+H). ¹H NMR (400 MHz, CDCl₃) δ ppm 7.50 (s, 1H), 7.30 (m, 2H), 7.06-7.02 (m, 2H), 6.89 (s, 1H), 4.79 (d, J=16 Hz, 1H), 4.66-4.59 (m, 2H), 4.42 (d, J=16 Hz, 1H), 4.36 (d, J=16 Hz, 1H), 2.49-2.41 (m, 1H), 1.10 (d, J=8.00 Hz, 3H), 0.83 (d, J=8.00 Hz, 3H). Resolution of the enantiomers is achieved by chiral HPLC using the ChiralPak IA column with 20% i-PrOH/Hexanes as mobile phase to give enantiomers with t, 32 min and tᵣ=41 min.

### 7-(3-Fluoro-benzyl)-5-isopropyl-7,8-dihydro-imidazo[1,5-a]pyrazin-6-one

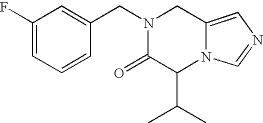

MS (ESI) m/z 288.2 (M+H). ¹H NMR (400 MHz, CDCl₃) δ ppm 7.47 (s, 1H), 7.34-6.97 (m, 4H), 6.88 (s, 1H), 4.80 (d, J=16 Hz, 1H), 4.66-4.63 (m, 2H), 4.46 (d, J=16 Hz, 1H), 4.39 (d, J=16 Hz, 1H), 2.48-2.40 (m, 1H), 1.11 (d, J=4.00 Hz, 3H), 0.85 (d, J=4.00 Hz, 3H). Resolution of the enantiomers is achieved by chiral HPLC using the ChiralPak IA column with 20% i-PrOH/Hexanes as mobile phase to give enantiomers with tᵣ=31 min and tᵣ=41 min.

### 7-Benzyl-5-isopropyl-7,8-dihydro-imidazo[1,5-a]pyrazin-6-one

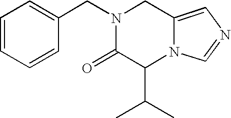

MS (ESI) m/z 270.3 (M+H). ¹H NMR (400 MHz, CDCl₃) δ ppm 7.45 (s, 1H), 7.36-7.27 (m, 5H), 6.85 (s, 1H), 4.84 (d, J=16 Hz, 1H), 4.64-4.60 (m, 2H), 4.41 (d, J=16 Hz, 1H), 4.36 (d, J=16 Hz, 1H), 2.48-2.40 (m, 1H), 1.09 (d, J=8.00 Hz, 3H), 0.83 (d, J=8.00 Hz, 0.3H).

### 5-Isopropyl-7-(3-methyl-benzyl)-7,8-dihydro-imidazo[1,5-a]pyrazin-6-one

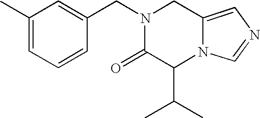

MS (ESI) m/z 284.0 (M+H). ¹H NMR(HCl salt, 400 MHz, DMSO-d₆) δ ppm 0.88 (d, J=6.82 Hz, 3H), 0.99 (d, J=6.82 Hz, 3H), 2.29 (s, 3H), 2.32-2.45 (m, 1H), 4.46-4.79 (m, 4H), 5.00 (d, J=6.06 Hz, 1H), 7.08-7.12 (m, 2H), 7.11 (m, 1H), 7.13 (s, 1H), 7.23-7.27 (m, 1H), 7.62 (s, 1H), 9.23 (s, 1H). Resolution of the enantiomers is achieved by chiral HPLC using the ChiralPak IA column with a 40% i-PrOH/hexanes as mobile phase to give enantiomers with retention time tᵣ=14 min and tᵣ=17 min.

### 7-(2-Chloro-benzyl)-5-isopropyl-7,8-dihydro-imidazo[1,5-a]pyrazin-6-one

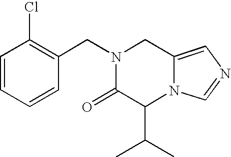

MS (ESI) m/z 304.1 (M+H). ¹H NMR (400 MHz, CDCl₃) δ ppm 7.48 (s, 1H), 7.40-7.21 (m, 4H), 6.88 (s, 1H), 4.91 (d, J=16 Hz, 1H), 4.86 (d, J=16 Hz, 1H), 4.65 (d, J=4.00 Hz, 1H), 4.49 (d, J=16 Hz, 1H), 4.43 (d, J=16 Hz, 1H), 2.48-2.39 (m, 1H), 1.1.1 (d, J=8.00 Hz 3H), 0.87 (d, J=8.00 Hz, 3H).

### 7-(2-Chloro-4-fluoro-benzyl)-5-isopropyl-7,8-dihydro-imidazo[1,5-a]pyrazin-6-one

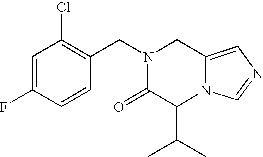

MS (ESI) m/z 322.1 (M+H). ¹H NMR (400 MHz, CDCl₃) δ ppm 0.95 (d, J=6.82 Hz, 3H) 1.13 (d, J=6.82 Hz, 3H) 2.54 (br s, 1H) 4.44-4.68 (m, 2H) 4.81 (d, J=14.65 Hz, 1H) 4.96 (d, J=14.65 Hz, 1H) 5.07 (br s, 1H) 7.02 (t, J=8.21 Hz, 1H) 7.18 (dd, J=8.21, 2.40 Hz, 1H) 7.37 (br s, 1H) 7.40-7.48 (m, 1H) 9.37 (br s, 1H). Resolution of the enantiomers is achieved by chiral HPLC using the ChiralPak IA column with a 30% i-PrOH/hexanes as mobile phase to give enantiomers with retention time tᵣ=21 min and tᵣ=24.5 min.

### 7-(4-Chloro-benzyl)-5-isopropyl-7,8-dihydro-imidazo[1,5-a]pyrazin-6-one

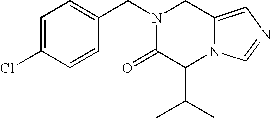

MS (ESI) m/z 304.0 (M+H). ¹H NMR (400 MHz, CDCl₃) δ ppm 0.83 (d, J=6.82 Hz, 3H), 1.10 (d, J=6.82 Hz, 3H), 2.35-2.53 (m, 1H), 4.39 (q, J=15.24 Hz, 2H), 4.55-4.69 (m, 2 H), 4.78 (d, 1H), 6.88 (s, 1H), 7.23 (d, 2H), 7.32 (d, 2H), 7.47 (s, 1H). Resolution of the enantiomers is achieved by chiral HPLC using the ChiralPak IA column with a 32% i-PrOH/hexanes as mobile phase to give enantiomers with retention time tᵣ=20.6 min and tᵣ=25.8 min.

### 7-(2,4-difluoro-benzyl)-5-isopropyl-7,8-dihydro-imidazo[1,5-a]pyrazin-6-one

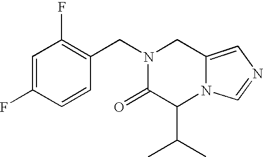

MS (ESI) m/z 306.0 (M+H). ¹H NMR (400 MHz, CDCl₃) δ ppm 0.92 (di J=6.57 Hz, 3H), 1.08 (d, J=6.82 Hz, 3H), 2.38-2.61 (m, 1H), 4.46-4.67 (m, 3H), 4.94 (d, J=14.65 Hz, 1H), 5.05 (d, J=4.80 Hz, 1H), 6.80-6.99 (m, 2H), 7.38 (s, 1H), 7.41-7.50 (m, 1H), 9.35 (s, 1H). Resolution of the enantiomers is achieved by chiral HPLC using the ChiralPak IA column with a 30% i-PrOH/hexanes as mobile phase to give enantiomers with tᵣ=19.3 min and tᵣ=24.1 min.

### 7-(4-Bromo-benzyl)-5-isopropyl-7,8-dihydro-imidazo[1,5-a]pyrazin-6-one

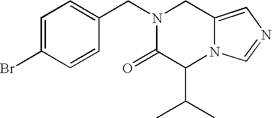

MS (ESI) m/z 349.9 (M+H). ¹H NMR (400 MHz, CDCl₃) δ ppm 0.83 (d, J=6.82 Hz, 3H), 1.10 (d, J=6.82 Hz, 3H), 2.37-2.51 (m, 1H), 4.39 (q, J=15.33 Hz, 2H), 4.54-4.67 (m, 2H), 4.71-4.81 (m, 1H), 6.88 (s, 1H), 7.18 (d, J=8.34 Hz, 2H), 7.42-7.52 (m, 3H). Resolution of the enantiomers is achieved by chiral HPLC using the ChiralPak IA column with a 30% i-PrOH/hexanes as mobile phase to give enantiomers with tᵣ=15.3 min and tᵣ=19.4 min.

### 7-(3-Chloro-4-fluoro-benzyl)-5-isopropyl-7,8-dihydro-imidazo[1,5-a]pyrazin-6-one

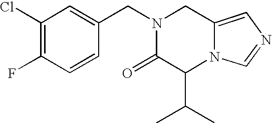

MS (ESI) m/z 322.2 (M+H). ¹H NMR (400 MHz, CDCl₃) δ ppm 7.47 (s, 1H), 7.38-7.10 (m, 3H), 6.90 (s, 1H), 4.72 (d, J=16 Hz, 1H), 4.65-4.60 (m, 2H), 4.46 (d, J=16 Hz, 1H), 4.37 (d, J=16 Hz, 1H), 2.49-2.41 (m, 1H), 1.11 (d, J=4.00 Hz, 3H), 0.83 (d, J=4.00 Hz, 3H).

### 7-(3,4-Difluoro-benzyl)-5-isopropyl-7,8-dihydro-imidazo[1,5-a]pyrazin-6-one

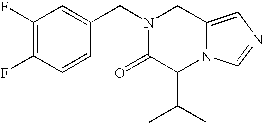

¹H NMR (400 MHz, CDCl₃) δ ppm 7.47 (s, 1 HO, 7.17-7.03 (m, 3H), 6.89 (s, 1H), 4.74 (d, J=12 Hz, 1H), 4.66-4.60 (m, 2H), 4.47 (d, J=12 Hz, 1H), 4.38 (d, J=12 Hz, 1H), 2.48-2.40 (m, 1H), 1.11 (d, J=8 Hz, 3H), 0.84 (d, J=8 Hz, 3H).

### 7-(4-trifluoromethyl-benzyl)-5-isopropyl-7,8-dihydro-imidazo[1,5-a]pyrazin-6-one

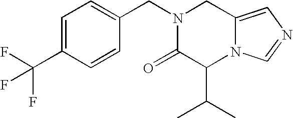

MS (ESI) m/z 338.1 (M+H). ¹H NMR (corresponding HCl salt, 400 MHz, DMSO-d₆) δ ppm 0.87 (d, J=6.82 Hz, 3H) 0.99 (d, J=6.82 Hz, 3H) 2.30-2.42 (m, 1H) 4.57-4.73 (m, 2H) 4.73-4.84 (m, 2H) 4.97 (d, J=6.06 Hz, 1H) 7.52 (d, J=7.83 Hz, 2H) 7.56 (s, 1H) 7.73 (d, J=8.08 Hz, 2H) 9.07 (s, 1H). Resolution of the enantiomers is achieved by chiral HPLC using the ChiralPak IA column with a 30% i-PrOH/hexanes as mobile phase to give enantiomers with tᵣ=20.5 min and tᵣ=25.5 min.

### 5-tert-Butyl-7-(4-chloro-benzyl)-7,8-dihydro-imidazo[1,5-a]pyrazin-6-one

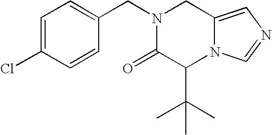

MS (ESI) m/z 318.0 (M+H). ¹H NMR (400 MHz, CDCl₃) δ ppm 1.03 (s, 9H) 4.31 (dc, 1H) 4.42 (d, 1H) 4.48 (s, 1H) 4.57 (d, J=14.40 Hz, 1H) 4.80 (d, J=14.40 Hz, 1H) 6.86 (s, 1H). 7.23 (d; 2H) 7.32 (d, 2H) 7.45 (s, 1H). Resolution of the enantiomers is achieved by chiral HPLC using the ChiralPak IA column with a 10% EtOH/acetonitrile as mobile phase to give enantiomers with tᵣ=22 min and tᵣ=28 min.

### 5-tert-Butyl-7-(4-chloro-3-fluoro-benzyl)-7,8-dihydro-imidazo[1,5-a]pyrazin-6-one

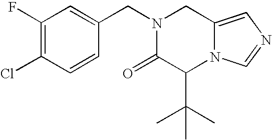

MS (ESI) m/z 336.1 (M+H). ¹H NMR (400 MHz, CDCl₃) δ ppm 1.04 (s, 9H) 4.24-4.39 (m, 1H) 4.43-4.54 (m, 2H) 4.56-4.66 (m, 1H) 4.68-4.80 (m, 1H) 6.88 (s, 1H) 7.02 (dd, J=8.21, 1.39 Hz, 1H) 7.10 (dd, J=9.47, 1.89 Hz, 1H) 7.37 (t, J=7.83 Hz, 1H) 7.46 (s, 1H). Resolution of the enantiomers is achieved by chiral HPLC using the ChiralPak AS-H column with a 20% EtOH/Hexanes as mobile phase to give enantiomers with tᵣ, 14.7 min and tᵣ=28.5 min.

### 5-tert-Butyl-7-(3,4-difluoro-benzyl)-7,8-dihydro-imidazo[1,5-a]pyrazin-6-one

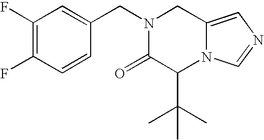

MS (ESI) m/z 320.3 (M+H). ¹H NMR (400 MHz, CDCl₃) δ ppm 7.47 (s, 1H), 7.17-7.05 (m, 3H), 6.88 (s, 1H), 4.745 (d, J=12 Hz, 1H), 4.595 (d, J=12 Hz, 1H), 4.50 (s, 1H), 4.48 (d, J=16 Hz, 1H), 4.36, (d, J=16 Hz, 1H), 1.04 (s, 9H).

### 4-(5-tert-Butyl-6-oxo-5,6-dihydro-8H-imidazo[1,5-a]pyrazin-7-ylmethyl)-benzonitrile

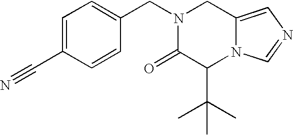

MS (ESI) m/z 309.1 (M+H). ¹H NMR (400 MHz, CDCl₃) δ ppm 1.04 (s, 9H) 4.24-4.41 (m, 1H) 4.42-4.57 (m, 2H) 4.76 (s, 2H) 6.88 (s, 1H) 7.39 (d, J=8.34 Hz, 2H) 7.46 (s, 1H) 7.64 (d, J=8.34 Hz, 2H). Resolution of the enantiomers is achieved by chiral HPLC using the ChiralPak AS-H column with 35% EtOH/Hexanes as mobile phase to give enantiomers with tᵣ=17.8 min and tᵣ=30 min.

### 7-(4-Chloro-benzyl)-5-cyclopropyl-7,8-dihydro-imidazo[1,5-a]pyrazin-6-one

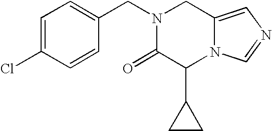

MS (ESI) m/z 302.0 (M+H). ¹H NMR (based on HCl salt, 400 MHz, DMSO-d₆) δ ppm 0.58 (d, J=21.47. Hz, 1H) 0.66-0.82 (m, 3H), 1.43-1.54 (m, 1H), 4.51 (d, J=9.60 Hz, 1H), 4.60-4.67 (m, 2H), 4.73 (d, 2H), 7.32 (d, J=8.34 Hz, 2H), 7.43 (d, 2H), 7.58 (s, 1H), 9.18 (s, 1H). Resolution of the enantiomers is achieved by chiral HPLC using the ChiralPak IA column with 5% EtOH/Acetonitrile as mobile phase to give enantiomers with tᵣ=12 min and tᵣ=13.5 min.

### 7-(4-fluoro-benzyl)-5-cyclopropyl-7,8-dihydro-imidazo[1,5-a]pyrazin-6-one

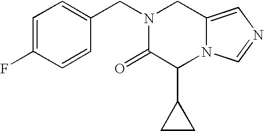

MS (ESI) m/z 286 (M+H). ¹H NMR (400 MHz, CDCl₃) δ ppm 0.30-0.47 (m, 1H) 0.58-0.74 (m, 2H) 0.74-0.85 (m, 1H) 1.23-1.40 (m, 1H) 4.29 (d, J=7.58 Hz, 1H) 4.33-4.53 (m, 2H) 4.70 (s, 2H) 6.87 (s, 1H) 7.03 (t, J=8.59 Hz, 2H) 7.18-7.37 (m, 2H) 7.53 (s, 1H). Resolution of the enantiomers is achieved by chiral HPLC using the ChiralPak AS-H column with 30% EtOH/Heptane as mobile phase to give enantiomers with tᵣ=12.5 min and tᵣ=15.0 min.

### 7-(4-methoxy-benzyl)-5-cyclopropyl-7,8-dihydro-imidazo[1,5-a]pyrazin-6-one

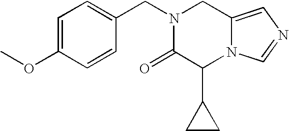

MS (ESI) m/z 298.3 (M+H). ¹H NMR (400 MHz, CDCl₃) δ ppm 0.28-0.44 (m, 1H) 0.58-0.73 (m, 2H) 0.74-0.84 (m, 1H) 1.26-1.36 (m, 1H) 3.79 (s, 3H) 4.27 (d, J=7.83 Hz, 1H) 4.31-4.48 (m, 2H) 4.58-4.73 (m, 2H) 6.78-6.93 (m, 3H) 7.20 (d, J=8.59 Hz, 2H) 7.51 (s, 1H).

### 4-(5-Cyclopropyl-6-oxo-5,6-dihydro-8H-imidazo[1,5-a]pyrazin-7-ylmethyl)-benzonitrile

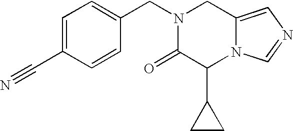

MS (ESI) m/z 293.2 (M+H); ¹H NMR (400 MHz, CDCl₃) δ ppm 0.29-0.47 (m, 1H) 0.56-0.74 (m, 2H) 0.75-0.86 (m, 1H) 1.25-1.39 (m, 1H) 4.29 (d, J=7.58 Hz, 1H) 4.38 (d; 1H) 4.52 (d, 1H) 4.66-4.87 (m, 2H) 6.87 (s, 1H) 7.37 (d, J=8.59 Hz, 2H) 7.54 (s, 1H) 7.63 (d, J=8.34 Hz, 2H). Resolution of the enantiomers is achieved by chiral HPLC using the ChiralPak AD-H column with 50% EtOH/Heptane as mobile phase to give enantiomers with tᵣ=25.3 min and tᵣ=41 min.

### 5-Cyclopropyl-7-(3,4-difluoro-benzyl)-7,8-dihydro-imidazo[1,5-a]pyrazin-6-one

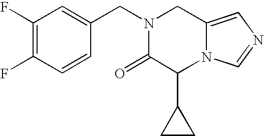

MS (ESI) m/z 304.3 (M+H). ¹H NMR (400 MHz, CDCl₃) δ ppm 0.28-0.47 (m, 1H) 0.57-0.71° (m, 2H) 0.73-0.84 (m, 1H) 1.12-1.35 (m, 1H) 4.24 (d, J=7.83 Hz, 1H) 4.39-4.61 (m, 2H) 4.74 (s, 2H) 6.75-6.96 (m, 3H) 7.28-7.41 (m, 1H) 7.53 (s, 1H). Resolution of the enantiomers is achieved by chiral HPLC using the ChiralPak AS-H column with 40% EtOH/Hexanes as mobile phase to give enantiomers with tᵣ=10 min and tᵣ=17 min.

### 7-(4-Chloro-3-fluoro-benzyl)-5-cyclopropyl-7,8-dihydro-imidazo[1,5]-pyrazin-6-one

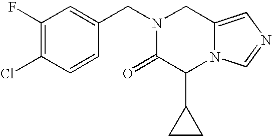

MS (ESI) m/z 320.1 (M+H). ¹H NMR (400 MHz, CDCl₃) δ ppm 0.28-0.46 (m, 1H) 0.56-0.73 (m, 2H) 0.74-0.88 (m, 1H) 1.23-1.36 (m, 1H) 4.28 (d, J=7.83 Hz, 1H) 4.32-4.57 (m, 2H) 4.68 (d, 2H) 6.86 (s, 1H) 7.00 (d, J=8.34 Hz, 1H) 7.07 (dd, J=9.47, 1.89 Hz, 1H). 7.35 (t, J=7.83 Hz, 1H) 7.53 (s, 1H). Resolution of the enantiomers is achieved by chiral HPLC using the ChiralPak AS-H column with 35% i-PrOH/Heptane as mobile phase to give enantiomers with tᵣ=22.3 min and tᵣ=28.3 min.

### 5-Cyclobutyl-7-(4-fluoro-benzyl)-7,8-dihydro-imidazo[1,5-a]pyrazin-6-one

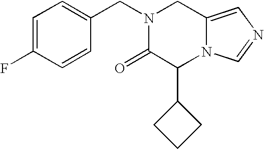

MS (ESI) m/z 300.0 (M+H). ¹H NMR (400 MHz, CDCl₃) δ ppm 1.63-1.79 (m, 2H) 1.79-1.95 (m, 2H) 1.96-2.13 (m, 2H) 2.72-2.89 (m, 1H) 4.24-4.43 (m, 2H) 4.53 (d, J=14.65 Hz, 1H) 4.65 (d, J=7.07 Hz, 1H) 4.75 (d, J=14.65 Hz, 1H) 6.85 (s, 1H) 6.93-7.07 (m, 2H) 7.13-7.25 (m, 2H) 7.46 (s, 1H). Resolution of the enantiomers is achieved by chiral HPLC using the ChiralPak AS-H column with 30% EtOH/Heptane as mobile phase to give enantiomers with tᵣ=18.5 min and tᵣ=22.1 min.

### 7-(4-fluoro-benzyl)-5-cyclopentyl-7,8-dihydro-imidazo[1,5-a]pyrazin-6-one

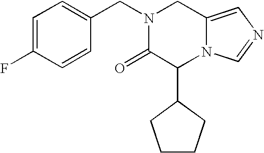

MS (ESI) m/z 314.0 (M+H). ¹H NMR (based on HCl salt, 400 MHz, DMSO-d₆) δ ppm 1.28-1.54 (m, 5H) 1.54-1.76 (m, 3H) 2.29-2.46 (m, 1H) 4.51-4.66 (m, 3H) 4.67-4.76 (m, 1H) 4.99 (d, J=8.34 Hz, 1H) 7.09-7.26 (m, 2H) 7.27-7.40 (m, 2H) 7.56 (s, 1H) 9.11 (s, 1H) Resolution of the enantiomers is achieved by chiral HPLC using the ChiralPak IA column with 50% i-PrOH/Hexanes as mobile phase to give enantiomers with tᵣ=17.5 min and tᵣ=21.5 min.

### 7-(4-Chloro-benzyl)-5-cyclohexyl-7,8-dihydro-imidazo[1,5-a]pyrazin-6-one

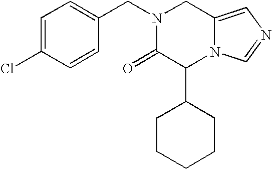

MS (ESI) m/z 344.2 (M+H). ¹H NMR (400 MHz, CDCl₃) δ ppm 0.80-0.96 (m, 1H) 0.97-1.12 (m, 1H) 1.24 (d, J=68.46 Hz, 3H) 1.43-1.86 (m, 5H) 1.98-2.16 (m, 1H) 4.15-4.50 (m, 2H) 4.55-4.80 (m, 3H) 6.86 (s, 1H) 7.21 (d, J=8.34 Hz, 2H) 7.32 (d, J=8.34 Hz, 2H) 7.45 (s, 1H). Resolution of the enantiomers is achieved by chiral HPLC using the ChiralPak IA column with 3% MeOH/CH₂Cl₂ as mobile phase to give enantiomers with tᵣ=12.75 min and tᵣ=15 min.

### 7-(4-Chloro-benzyl)-5-(tetrahydro-pyran-4-yl)-7,8-dihydro-imidazo[1,5-a]pyrazin-6-one

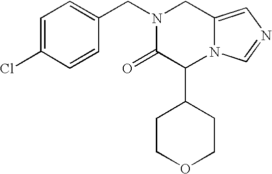

MS (ESI) m/z 346.0 (M+H). ¹H NMR (400 MHz, CDCl₃) δ ppm 1.29-1.45 (m, 2H) 1.52-1.70 (m, 2H) 2.10-2.30 (m, 1H) 3.24-3.42 (m, 2H) 3.84-4.06 (m, 2H) 4.30-4.49 (m, 2H) 4.60-4.67 (m, 1H) 4.69 (d, J=4.55 Hz, 2H) 6.88 (s, 1H) 7.21 (d, 2H) 7.32 (d, J=8.59 Hz, 2H) 7.47 (s, 1H). Resolution of the enantiomers is achieved by chiral HPLC using the ChiralPak IA column with 30% EtOH/Hexanes as mobile phase to give enantiomers with t, 21.4 min and tᵣ=30.5 min.

### 5-Isopropyl-7-pyridin-4-ylmethyl-7,8-dihydro-imidazo[1,5-a]pyrazin-6-one

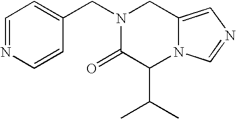

MS (ESI) m/z 271.2 (M+H). ¹H NMR (400 MHz, CDCl₃) δ ppm 8.59 (d, J=8.00 Hz, 2H), 7.50 (s, 1H), 7.19 (d, J=8.00 Hz, 2H), 6.90 (s, 1H), 4.78-4.67 (m, 3H), 4.53 (d, J=16.0 Hz, 1H), 4.39 (d, J=16.0 Hz, 1H), 2.49-2.41 (m, 1H), 1.12 (d, J=4.00 Hz, 3H), 0.87 (d, J=4.00 Hz, 3H).

### 7-(3,5-Dimethyl-benzyl)-5-isopropyl-7,8-dihydro-imidazo[1,5-a]pyrazin-6-one

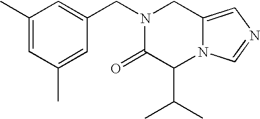

MS (ESI) m/z 298.2 (M+H). ¹H NMR (400 MHz, CDCl₃) δ ppm 7.45 (s, 1H), 6.93 (s, 1H), 6.89 (s, 2H, overlap), 6.86 (s, 1H), 4.76-4.55 (m, 3H), 4.36 (s, 2H), 2.48-2.44 (m, 1H), 2.29 (s, 6H), 1.11 (d, J=4.00 Hz, 3H), 0.84 (d, J=4.00 Hz, 3H).

### 7-Cyclohexylmethyl-5-isopropyl-7,8-dihydro-imidazo[5-a]pyrazin-6-one

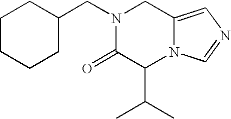

MS (ESI) m/z 276.3 (M+H). ¹H NMR (400 MHz, CDCl₃) δ ppm 7.46 (s, 1H), 6.92 (S, 1H), 4.61-4.54 (m, 3H), 4.42 (d, J=16 Hz, 1H), 3.36 (d, J=8.00Hz, 1H), 2.42-2.33 (m, 1H), 1.77-0.87 (m, 16H). Resolution of the enantiomers is achieved by chiral HPLC using the ChiralPak IA column with 25% i-PrOH/Hexanes as mobile phase to give enantiomers with tᵣ=15 min and tᵣ=30 min.

### 5-Isopropyl-7-(tetrahydro-pyran-4-ylmethyl)-7,8-dihydro-imidazo[1,5-a]pyrazin-6-one

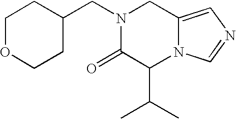

MS (ESI) m/z 278.3 (M+H). ¹H NMR (HCl salt, 400 MHz, MeOD) δ ppm 9.11 (s, 1H), 7.61 (s, 1H), 4.91 (d, J=4.00 Hz, 1H), 3.95-3.91 (m, 2H), 4.81 (s, 2H), 3.57-3.30 (m, 5H), 2.46-2.38 (m, 1H), 2.09-2.01 (m, 1H), 1.60-1.57 (m, 2H), 1.41-1.29 (m, 2H), 1.12 (d, J=6.8 Hz, 3H), 0.99 (d, J=6.8 Hz, 3H).

### 7-Cyclohexylmethyl-5 (1,1-dimethyl-propyl)-7,8-dihydro-imidazo[1,5-a]pyrazin-6-one

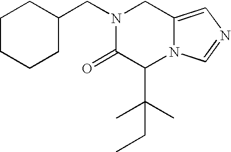

MS (ESI) m/z 304.2 (M+H). ¹H NMR (400 MHz, CDCl₃) δ ppm 0.88-1.03 (m, 11H) 1.10-1.29 (m, 4H) 1.31-1.44 (m, 1H) 1.43-1.54 (m, 1H) 1.60-1.78 (m, 5H) 3.26-3.41 (m, 2H) 4.36 (d, J=15.66 Hz, 1H) 4.53 (s, 1H) 4.59 (d, J=15.41 Hz, 1H) 6.91 (s, 1H) 7.43 (s, 1H). Resolution of the enantiomers is achieved by chiral HPLC using the ChiralPak IA column with 30% EtOH/Hexanes as mobile phase to give enantiomers with tᵣ=23.1 min and tᵣ=32 min.

### 4-[5-(1,1Dimethyl-propyl)-6-oxo-5,6-dihydro-8H-imidazo[15-a]pyrazin-7-ylmethyl]-benzonitrile

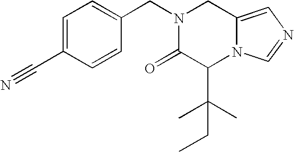

MS (ESI) m/z 323.3 (M+H). ¹H NMR (400 MHz, CDCl₃) δ ppm 0.88-1.02 (m, 9H) 1.33-1.51 (m, 2H) 4.34 (d, 1H) 4.51 (d, 1H) 4.63 (s, 1H) 4.68-4.84 (m, 2H) 6.92 (s, 1H) 7.39 (d, J=8.08 Hz, 2H) 7.57 (s, 1H) 7.64 (d, J=8.34 Hz, 2H). Resolution of the enantiomers is achieved by chiral HPLC using the ChiralPak AS-H column with 30% i-PrOH/Hexanes as mobile phase to give enantiomers with tᵣ=27 min and tᵣ=56 min.

### 5-(1,1-Dimethyl-propyl)-7-(tetrahydro-pyran-4-ylmethyl)-7,8-dihydro-imidazo[5-a]pyrazin-6-one

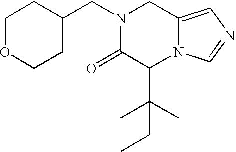

MS (ESI) m/z 306.2 (M+H). ¹H NMR (400 MHz, CDCl₃) δ ppm 7.48 (s, 1H), 6.91 (s, 1H), 4.63 (d, J=16 Hz, 1H), 4.54 (s, 1H), 4.38 (d, J=16 Hz, 1H), 3.98-3.95 (m, 2H), 3.49-3.31 (m, 4H), 2.01-1.95 (m, 1H), 1.55-0.90 (m, 1-5H). Resolution of the enantiomers is achieved by chiral HPLC using the ChiralPak AS-H column with 15% EtOH/Heptane as mobile phase to give enantiomers with tᵣ=9.5 min and tᵣ=14.3 min.

### 5-(1,1-Dimethyl-propyl)-7-(4-fluoro-benzyl)-7,8-dihydro-imidazo[1,5-a]pyrazin-6-one

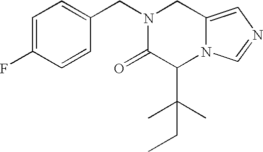

MS (ESI) m/z 316.3 (M+H). ¹H NMR (based on the corresponding HCl salt, 400 MHz, MeOD) δ ppm 9.06 (s, 1H), 7.57 (s, 1H), 7.42 (m, 2H), 7.09 (m, 2H), 5.01-4.63 (m, 5H), 1.51-0.89 (m, 11H). Resolution of the enantiomers is achieved by chiral HPLC using the ChiralPak AS-H column with 23% EtOH/Heptane as mobile phase to give enantiomers with tᵣ=9.55 min and tᵣ=16.34 min.

### 5-(1-Cyclopropyl-1-methyl-ethyl)-7-(1-hydroxy-cyclohexylmethyl)-7,8-dihydro-imidazo[1,5-a]pyrazin-6-one

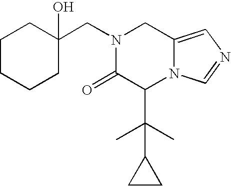

MS (ESI) m/z 332.3 (M+H). ¹H NMR (400 MHz, CDCl₃) δ ppm 0.15-0.26 (m, 1H) 0.26-0.35 (m, 1H) 0.33-0.43 (m, 2H), 0.64-0.79 (m, 1H) 0.93 (d, J=16.67 Hz, 6H) 1.20-1.37 (m, 1H) 1.37-1.49 (m, 2H) 1.50-1.65 (m, 7H) 3.54 (d, J=4.55 Hz, 2H) 4.55 (s, 1. H) 4.58-4.68 (m, 1H) 4.71-4.85 (m, 1H) 6.89 (s, 1H) 7.50 (s, 1H).

### 7-Cyclopropylmethyl-5-isopropyl-7,8-dihydro-imidazo[1,5-a]pyrazin-6-one

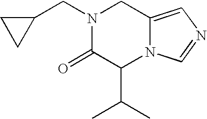

MS (ESI) m/z 234.3 (M+H). ¹H NMR (400 MHz, CDCl₃) δ ppm 7.16 (s, 1H), 6.64 (s, 1H), 4.41 (d, J=16 Hz, 1H), 4.28 (d, J=4.00 Hz, 1H), 4.23 (d, J=16 Hz, 1H), 3.44-3.39 (m, 1H), 2.88-2.83 (m, 1H), 2.18-2.10 (m, 1H), 0.81 (d, J=4.00 Hz, 3H), 0.78-0.71 (m, 1H), 0.59 (d, J=4.00 Hz, 1H), 0.30-0.22 (m, 2H), 0.08-0.0 (m, 2H).

### 5-(1-Cyclopropyl-1-methyl-ethyl)-7-(4-fluoro-benzyl)-7,8-dihydro-imidazo[1,5-a]pyrazin-6-one

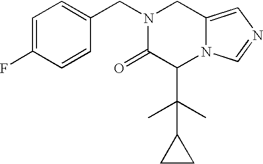

MS (ESI) m/z 328.2 (M+H). ¹H NMR (400 MHz, CDCl₃) δ ppm 7.30 (S, 1H), 7.09-7.06 (m, 2H), 6.84-6.79 (m, 2H), 6.65 (s, 1H), 4.51 (d, J=16 Hz, 1H), 4.42 (d, J=16 Hz, 1H), 4.37 (s, 1H), 4.28 (d, J=16 Hz, 1H), 4.13 (d, J=16 Hz, 1H), 1.04-0.98 (m, 1H), 0.74 (s, 3H), 0.64 (s, 3H), 0.45-0.39 (m, 1H), 0.13-0.10 (m, 3H). Resolution of the enantiomers is achieved by chiral HPLC using the ChiralPak AS-H column with 15% EtOH/Heptane as mobile phase to give enantiomers with tᵣ=33 min and tᵣ=76 min.

### 4-[5-(1-Cyclopropyl-1-methyl-ethyl)-6-oxo-5,6-dihydro-8H-imidazo[1,5-a]pyrazin-7-ylmethyl]-benzonitrile

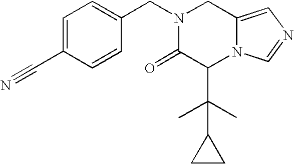

MS (ESI) m/z 335.2 (M+H). ¹H NMR (400 MHz, CDCl₃) δ ppm 0.13-0.38 (m, 4H) 0.55-0.70 (m, 1H) 0.85 (s, 0.3H) 0.95 (s, 3H) 4.33 (d, J=15.16 Hz, 1H) 4.48-4.70 (m, 3H) 4.85 (d, J=15.16 Hz, 1H) 6.86 (s, 1H) 7.38 (d, J=8.34 Hz, 2H) 7.51 (s, 1H) 7.62 (d, J=8.34 Hz, 2H). Resolution of the enantiomers is achieved by chiral HPLC using the ChiralPak AS-H column with 40% EtOH/Heptane as mobile phase to give enantiomers with tᵣ=11.2 min and tᵣ=20.5 min.

### 5-(1-Cyclopropyl-1-methyl-ethyl)-7-(tetrahydro-pyran-4-ylmethyl)-7,8-dihydro-imidazo[1,5-a]pyrazin-6-one

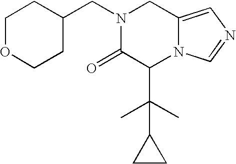

MS (ESI) m/z 318.4 (M+H). ¹H NMR (400 MHz, CDCl₃) δ ppm 0.19-0.26 (m, 1H) 026-0.33 (m, 1H) 0.34-0.40 (m, 2H) 0.63-0.74 (m, 1H) 0.89 (s, 3H) 0.95 (s, 3H) 1.34-1.46 (m, 2H) 1.50-1.62 (m, 2H) 3.22 (dd, J-13.39, 7.33 Hz, 1H) 3.30-3.41 (m, 2H) 3.55 (dd, J=13.52, 7.20 Hz, 1H) 3.91-4.08 (m, 3H) 4.39 (d, J=15.41 Hz, 1H) 4.53 (s, 1H) 4.67 (d, J=15.16 Hz, 1H) 6.92 (s, 1H) 7.51 (s, 1H). Resolution of the enantiomers is achieved by chiral HPLC using the ChiralPak AS-H column with 30% i-PrOH/Hexanes as mobile phase to give enantiomers With tᵣ=14.5 min and tᵣ=44 min.

### 7-(4-Fluoro-benzyl)-5-isopropyl-8-methyl-7,8-dihydro-imidazo[1,5-a]pyrazin-6-one

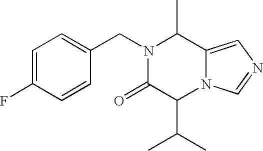

MS (ESI) m/z 302.1 (M+H). ¹H NMR (400 MHz, CDCl₃ with ˜10% minor diastereomer) δ ppm 1.08 (d, J=6.82 Hz, 3H) 1.22 (d, J=6.82 Hz, 3H) 1.62 (d, J=6.82 Hz, 3H) 1.65-1.71 (m, 1H) 2.42-2.52 (m, 1H) 4.17 (d, J=14.91 Hz, 1H) 4.76 (q, J=6.74 Hz, 1H) 4.96 (d, J=6.32 Hz, 1H) 5.35 (d, J=14.91 Hz, 1H) 6.99-7.08 (m, 2H) 7.20-7.25 (m, 2H) 7.32 (s, 1H) 9.16 (s, 1H).

### 7-(4-Fluoro-benzyl)-5,5-dimethyl-7,8-dihydro-imidazo[1,5-a]pyrazin-6-one

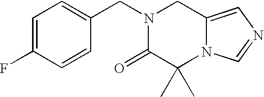

MS (ESI) m/z 274.0 (M+H). ¹H NMR (400 MHz, DMSO-d₆) δ ppm 1.77 (s, 6H), 4.68 (s, 2H), 4.69 (s, 2H), 7.15-7.26 (m, 2H), 7.27-7.41 (m, 2H), 7.59 (s, 1H), 9.34 (s, 1H).

### Example 4

### 4-(1,1-Dimethyl-propyl)-(3-fluoro-benzyl)-7,8-dihydro-6H-2,3a,6-triaza-azulen-5-one

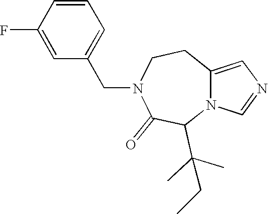

### A (1-Trityl-1H-imidazol-4-yl)acetic acid (Cas # 168632-03-9),

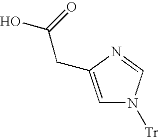

Trityl chloride (77 g, 0.276 mol) is added to a suspension of (1H-imidazol-4-yl)acetic acid hydrochloride (37.5 g, 0.23 mol) in pyridine (500 mL) at room temperature. This is stirred at room temperature for 16 h, at the end of which MeOH (200 mL) is added. This solution is stirred at room temperature for 1 h. Solvents were evaporated and the residue is taken up in CH₂Cl₂ and washed with 1 M aqueous citric acid solution (2×) and brine. The organic phase is dried over anhydrous Na₂SO₄ and evaporated to give a sticky residue which when taken up in diethyl ether and evaporated gave the product as a white solid that is used without further purification. MS (ESI) m/z 368.9 (M+H) (Procedure adapted from J. Org. Chem. 1993, 58, 4606, also prepared in WO2003013526)

### B. 2-(1-Trityl-1H-imidazol-4-yl)ethanol (Cas # 127607-62-9)

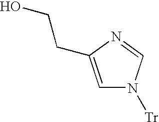

(1-Trityl-1H-imidazol-4-yl)acetic acid (41 g, 0.114 mol) is suspended in THF (400 mL) and cooled to 0° C. To this is added. BH₃.THF solution (222 mL, 1.0 M). The clear solution obtained is stirred at 0° C. for 1 h before warming to room temperature until LCMS indicates completion of the reaction. The solution is cooled again to 0° C. and quenched carefully with water (200 mL). The resulting solution is diluted with EtOAc (400 mL) and transferred to a separatory funnel and the aqueous layer is extracted with EtOAc (400 mL×3). The organic phase is dried over anhydrous Na₂SO₄ and evaporated to give a sticky residue which is taken up in ethanolamine (700 mL) and heated to 90° C. for 2.5 h. The reaction is transferred to a separatory funnel, diluted with EtOAc (1 L) and washed with water (3×600 mL). The organic phase is dried over anhydrous Na₂SO₄ and evaporated to give 2-(1-trityl-1H-imidazol-4-yl)-ethanol as a white solid that is used as is without further purification. MS (ESI) m/z 354.8 (M+H) (prepared by alternate method in J. Med. Chem. 1996, 39(19), 3806)

### C. Methanesulfonic acid 2-(1-trityl-1H-imidazol-4-yl)-ethyl ester

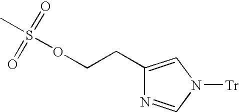

MsCl is added dropwise to a solution of 2-(1-trityl-1H-imidazol-4-yl)-ethanol (41 g, 115.7 mmol) and Et₃N (40.62 mL, 289.2 mmol) in CH₂Cl₂ at 0° C. The mixture is stirred for 1 h at 0° C., and then warmed to room temperature. After 1 h, the reaction is quenched with saturated NaHCO₃ (100 mL), and extracted with CH₂Cl₂ (400 mL×4). The combined organic layer is washed with brine, and dried over Na₂SO₄. After filtration and concentration, a solid is obtained and used as is in the next step.

### D. (3-Fluoro-benzyl)-[2-(1-trityl-1H-imidazol-4-yl)-ethyl]-amine

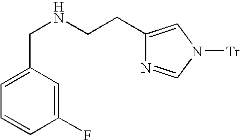

3-fluorobenzylamine (10.4 mL, 90.8 mmol) is added dropwise to a suspension of methanesulfonic acid 2-(1-trityl-1H-imidazol-4-yl)-ethyl ester (13.2 g, 30.2 mmol), K₂CO₃ (12.5 g, 90.8 mmol), NaI (13.61 g, 90.8 mmol) in DMF. The mixture is heated to 100° C. for 3 h. After filtration, the residue is: washed with CH₂Cl₂ (60 mL×3). The solvents are removed under vacuum. The residue is purified by flash column, and give a oil.

### E. 4-(1,1-Dimethyl-propyl)-6-(3-fluoro-benzyl)-7,8-dihydro-6H-2,3a,6-triaza-azulen-5-one

2-Bromo-3,3-dimethyl-pentanoyl chloride (650 mg, 2.85 mmol) is added dropwise to a solution of (3-fluoro-benzyl)-[2-(1-trityl-1H-imidazol-4-yl)-ethyl]-amine (1.1 g, 2.38 mmol) and Et₃N (1 mL, 37.14 mmol) in CH₂Cl₂ (17 mL) at 0° C. after 2 h, the solvent is removed under vacuum. 10 mL of saturated NaHCO₃ is added, and the mixture is extracted with CH₂Cl₂ (20 mL×4). The combined extracts are washed with brine, and dried over anhydrous Na₂SO₄. After filtration and concentration, a oily residue is obtained and dissolved in 15 mL of DMF and heated to 170° C. for 2 h by microwave. The solvent is removed, and the residue is dissolved into MeOH and heated to reflux for 2 h. After concentration, saturated NaHCO₃ solution is added. The mixture is extracted with CH₂Cl₂ (20 mL×4). The combined extracts are washed with brined, and dried over Na₂SO₄. After filtration and concentration, the residue is purified by flash column and give 342 mg of title compound as solid.

MS (ESI) m/z 330.2 (M+H). ¹H NMR (400 MHz, CDCl₃) δ ppm 7.36 (s, 1H), 7.32-7.27 (m, 1H), 7.06-6.96 (m, 3H), 6.81 (s, 1H), 5.02 (s, 1H), 4.93 (d, J=16 Hz, 1H), 4.36 (d, J=16 Hz, 1H), 3.70-3.65 (m, 1H), 3.41 (brs, 1H)ⱼ, 3.26-3.19 (m, 1H), 2.99-2.92 (m, 1H), 1.57-1.41 (m, 2H), 1.10 (s, 3H), 0.98-0.91 (m, 6H). Resolution of the enantiomers is achieved by chiral HPLC using the ChiralPak AS-H column with a 20% Ethanol/Hexanes as mobile phase to give enantiomers.

The following compounds can be prepared with similar method.

### 6-(3-Fluoro-benzyl)-4-isopropyl-7,8-dihydro-6H-2,3a,6-triaza-azulen-5-one

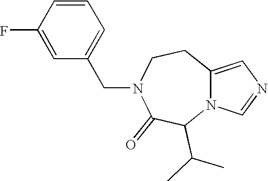

MS (ESI) m/z 302.2 (M+H). ¹H NMR (400 MHz, CDCl₃) δ ppm 7.38 (s, 1H), 7.33-7.27 (m, 1H), 7.05-6.97 (m, 3H), 6.83 (s, 1H), 4.92 (d, J=16 Hz, 1H), 4.58, (d, J=8 Hz, 1H), 4.35 (d, J=16 Hz, 1H), 3.92-3.85 (m, 11H), 3.47-3.41 (m, 1H), 3.10-2.92 (m, 2H), 1.15 (d, J=8 Hz, 3H), 0.90 (d, J=8 Hz, 3H).

### 6-(4-Fluoro-benzyl)-4-isopropyl-7,8-dihydro-6H-2,3a 6-triaza-azulen-5-one

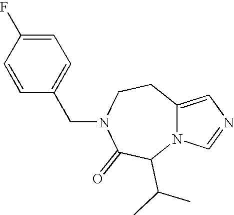

MS (ESI) m/z 302.1 (M+H). ¹H NMR (400 MHz, CDCl₃) δ ppm 7.36 (s, 1H), 7.26-7.23 (m, 2H), 7.04-6.99 (m, 2H), 6.81 (s, 1H), 4.85 (d, J 16 Hz, 1H), 4.57 (d, J=12 Hz, 1H), 4.37 (d, J=12 Hz, 1H), 3.91-3.83 (m, 1H), 3.47-3.40 (m, 1H), 3.07-2.88 (m, 2H), 2.40-2.25 (m, 1H), 1.14 (d, J=8 Hz, 3H), 0.89 (d, J=8 Hz, 3H).

### 4-Cyclobutyl-6-(4-fluoro-benzyl)-7,8-dihydro-6H-2,3a,6-triaza-azulen-5-one

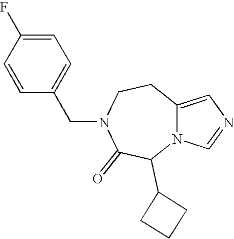

MS (ESI) m/z 314 (M+H). ¹H NMR (400 MHz, CDCl₃) δ ppm 7.27 (s, 1H), 7.13-7.09 (m, 2H), 6.96-6.90 (m, 2H), 6.71 (s, 1H), 4.92, (d, J=12 Hz, 1H), 4.62 (d, J=16 Hz, 1H), 4.41 (d, J=12 Hz, 1H), 3.79-3.71 (m, 1H), 3.53-3.47 (m, 1H), 3.17-3.10 (m, 1H), 2.88-2.85 (m, 2H), 2.34-2.13 (m, 2H), 1.98-1.88 (m, 4H). Resolution of the enantiomers is achieved by chiral HPLC using the ChiralPak IA column with a 25% Ethanol heptane as mobile phase to give enantiomers.

### 4-Cyclobutyl-6-(3-fluoro-benzyl)-7,8-dihydro-6H-2,3a,6-triaza-azulen-5-one

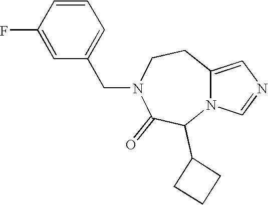

MS (ESI) m/z 314.1 (M+H). ¹H NMR (400 MHz, CDCl₃) δ ppm 7.35 (s, 1H), 7.31-7.26 (m, 1H), 7.00-6.96 (m, 2H), 6.93-6.90 (m, 1H), 6.80 (s, 1H), 5.01 (d, J=8.00 Hz, 1H), 4.69 (d, J=16 Hz, 1H), 4.53 (d, J=16 Hz, 1H), 3.86-3.79 (m, 1H), 3.62-3.56 (m, 1H), 3.27-3.17 (m, 1H), 3.05-2.96 (m, 2H), 2.36-2.20 (m, 2H), 2.08-1.93 (m, 4H).

### 4-(4-Cyclobutyl-5-oxo-4,5,7,8-tetrahydro-2,3a,6-triaza-azulen-6-ylmethyl)-benzonitrile

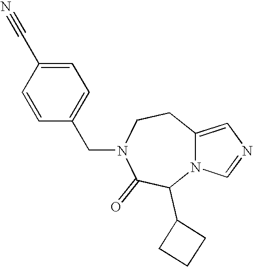

MS (ESI) m/z 321.1 (M+H). ¹H NMR (400 MHz, CDCl₃) δ ppm 7.62 (d, J=8.00 Hz, 2H), 7.36 (s, 1H), 7.31 (d, J=8.00 Hz, 2H), 6.82 (s, 1H), 5.02 (d, J=12 Hz, 1H), 4.77 (d, J=16 Hz, 1H), 4.57 (d, J=16 Hz, 0.1H), 3.90-3.83 (m, 1H), 3.62-3.55 (m, 1H), 3.28-3.19 (m, 1H), 3.03-2.96 (m, 2H), 2.35-2.20 (m, 2H), 2.10-1.90 (m, 4H).

### 4-(1,1-Dimethyl-propyl)-6-(4-fluoro-benzyl)-7,8-dihydro-6H-2,3a,6-triaza-azulen-5-one

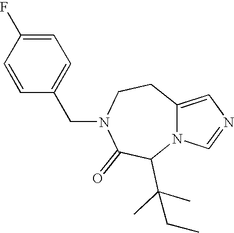

MS (ESI) m/z 330 (M+H). ¹H NMR (400 MHz, CDCl₃) δ ppm 7.35 (S, 1H), 7.28-7.25 (m, 2H), 7.05-6.99 (m, 2H), 6.81 (s, 1H), 5.00 (s, 1H), 4.89 (d, J=12 Hz, 1H), 4.35 (d, J=12 Hz, 1H), 3.70-3.65 (m, 1H), 3.39 (brs, 1H), 3.23-3.17 (m, 1H), 2.96-2.89 (m, 1H), 1.56-1.41 (m, 2H), 1.08-0.86 (m, 9H).

### Example 5

### 5-tert-Butyl-7-(4-fluoro-benzyl)-5-propyl-7,8-dihydro-imidazo[1,5-a]pyrazin-6-one

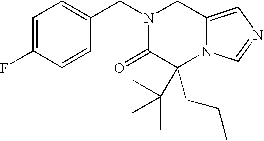

a solution of LiHMDS (3.6 mL, 1.0 M in THF) is added dropwise to a solution of 5-tert-Butyl-7-(4-fluoro-benzyl)-7,8-dihydro-imidazo[1,5-a]pyrazin-6-one (217 mg, 0.72 mmol) in 7 mL of dry THF at −78° C. The resulting mixture is stirred for 1 h at this temperature. Iodopropane (0.212 mL, 2.16 mmol) is added. The mixture is stirred for overnight and slowly warmed up to 0° C. Saturated NH₄Cl solution is added, and the mixture is extracted with CH₂Cl₂ (4×20 mL). The combined extracts are washed with brine and dried over anhydrous Na₂SO₄. After concentration, the residue is purified by flash column and yield 178 mg of solid.

MS (ESI) m/z 344.1 (M+H). ¹H NMR (400 MHz, CDCl₃) δ ppm 7.38 (s, 1H), 7.23-7.20 (m, 2H), 6.98-6.93 (m, 2H), 6.76 (s, 1H), 4.69 (d, J=16 Hz, 1H), 4.56 (d, J=16 Hz, 1H), 4.32 (s, 2H), 2.55-2.45 (m 1H), 1.90-1.80 (m, 1H), 0.90 (s, 9H), 1.10-0.60 (m, 2H), 0.81 (t, J=8.00 Hz, 3H). Resolution of the enantiomers is achieved by chiral HPLC using the ChiralPak IA column with 10% i-PrOH/Hexanes as mobile phase to give enantiomers with tᵣ=25 min and tᵣ=27 min.

The following compound can be prepared with similar method.

### 5-tert-Butyl-5-ethyl-7-(4-fluoro-benzyl)-7,8-dihydro-imidazo[1,5-a]pyrazin-6-one

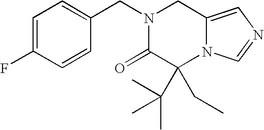

MS (ESI) m/z 330.3 (M+H). ¹H NMR (400 MHz, CDCl₃) δ ppm 7.46 (s, 1H), 7.31-7.28 (m, 2H), 7.05-7.01 (m, 2H), 6.84 (s, 1-H), 4.80 (d, J=16 Hz, 1H), 4.62 (d, J=16 Hz, 1H), 4.41 (s, 2H), 2.67-2.65 (m, 1H), 2.05-2.03 (m, 1H), 0.98 (s, 9H), 0.62 (t, J=8.00 Hz, 3H). Resolution of the enantiomers is achieved by chiral HPLC using the ChiralPak IA column with 10% i-PrOH/Hexanes as mobile phase to give enantiomers with tᵣ=23min and t, 26.5 min.

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A compound of formula (I): 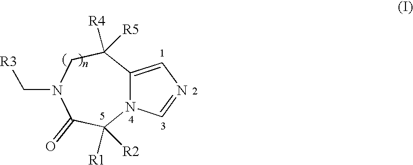
wherein
n is 0 or 1;
R₂ is hydrogen; or
R₁ and R₂ are independently alkyl, non-aromatic heterocyclyl, cycloalkyl, cycloalkyl-alkyl, alkenyl, or alkynyl; or
R₁ and R₂ together with the carbon atom to which they are attached optionally form a 3to 7-membered ring;
R₃ is heterocyclyl, alkyl, haloalkyl, aryl, or heteroaryl, each of which is optionally substituted with one to three substituents selected from alkyl, halogen, trifluoromethyl, cyano, alkoxy, cycloalkyl, hydroxy or cycloalkyl-alkyl;
R₄ and R₅ are independently hydrogen, halogen, hydroxy, or alkyl; or
a pharmaceutically acceptable salt thereof; or an optical isomer thereof; or a mixture of optical isomers.

2. The compound of claim 1, wherein n is 0 or 1; R₂ is hydrogen; or R₁ and R₂ are independently (C₁-C₇) alkyl, (4- to 9-membered)-non-aromatic heterocyclyl, (C₁-C₇) alkenyl, (C₁-C₇) alkynyl, (C₃-C₇) cycloalkyl, or (C₃-C₇) cycloalkyl-(C₁-C₇) alkyl; R₃ is (4- to 9-membered)-non-aromatic heterocyclyl, (C₁-C₇) alkyl, (C₁-C₇) haloalkyl, (C₃-C₇) cycloalkyl, (C₆-C₁₀) aryl or (C₆-C₁₀) heteroaryl, each of which is optionally substituted with one to three substituents selected from (C₁-C₇) alkyl, halogen, trifluoromethyl, cyano, (C₁-C₇) alkoxy, (C₃-C₇) cycloalkyl, or hydroxy; R₄ and R₅ are independently hydrogen, halogen, hydroxy, or (C₁-C₇) alkyl; or R₁ and R₂ together with the carbon atom to which they are attached optionally form a 3- to 7-membered ring; or a pharmaceutically acceptable salt thereof; or an optical isomer thereof; or a mixture of optical isomers.

3. The compound of claim 1, wherein R₂ is hydrogen; or R₁ and R₂ are independently (C₁-C₇) alkyl, (4- to 7-membered)-non-aromatic heterocyclyl, (C₃-C₇) cycloalkyl or (C₃-C₇) cycloalkyl-(C₁-C₇) alkyl; R₃ is (4- to 7-membered)-heterocyclyl, (C₁-C₇) alkyl, (C₁-C₇) haloalkyl, (C₃-C₇) cycloalkyl, (C₃-C₇) cycloalkyl-(C₁-C₇) alkyl, (C₆-C₁₀) aryl or (C₆-C₁₀) heteroaryl, each of which is optionally substituted with one to three substituents selected from (C₁-C₇) alkyl, halogen, trifluoromethyl, cyano, (C₁-C₇) alkoxy, (C₃-C₇) cycloalkyl, or hydroxy; R₄ and R₅ are independently hydrogen, or (C₁-C₇) alkyl; or R₁ and R₂ together with the carbon atom to which they are attached optionally form a 3- to 7-membered ring; or a pharmaceutically acceptable salt thereof; or an optical isomer thereof; or a mixture of optical isomers.

4. The compound of claim 1, wherein n is 0 or 1; R₁ is hydrogen or (C₁-C₇) alkyl; R₂ is (C₃-C₇) cycloalkyl, (C₃-C₇) cycloalkyl-(C₁-C₇) alkyl, or (C₁-C₇) alkenyl; R₃ is (4- to 7-membered)-heterocyclyl, (C₁-C₇) alkyl, (C₃-C₇) cycloalkyl, or (C₆-C₁₀) aryl, each of which is optionally substituted with one to three substituents selected from (C₁-C₇) alkyl, halogen, trifluoromethyl, cyano, (C₁-C₇) alkoxy, or hydroxy; R₄ and R₅ are independently hydrogen; or R₁ and R₂ together with the carbon atom to which they are attached optionally form a 3- to 7-membered ring; or pharmaceutically acceptable salts thereof; or an optical isomer thereof; or a mixture of optical isomers.

5. The compound of claim 1, wherein n is 0 or 1; R₁ is hydrogen or (C₁-C₇) alkyl; R₂ is (C₁-C₇) alkyl; R₃ is (C₃-C₇) cycloalkyl, or (C₆-C₁₀) aryl, each of which is optionally substituted with one to three substituents selected from (C₁-C₇) alkyl, halogen, trifluoromethyl, cyano, (C₁-C₇) alkoxy, or hydroxy; R₄ and R₅ are independently hydrogen; or R₁ and R₂ together with the carbon atom to which they are attached optionally form a 3- to 7-membered ring; or pharmaceutically acceptable salts thereof; or an optical isomer thereof; or a mixture of optical isomers.

6. A method of inhibiting aldosterone synthase activity in a subject in need thereof, comprising:
administering to the subject in need thereof a therapeutically effective amount of the compound according to claim 1.

7. A method of treating a disorder or a disease in a subject in need thereof mediated by aldosterone synthase, comprising:
administering to the subject a therapeutically effective amount of the compound according to claim 1.

8. The method of claim 7, wherein the disorder or disease in a subject is characterized by an abnormal activity of aldosterone synthase.

9. The method of claim 7, wherein the disorder or disease in a subject is characterized by an abnormal expression of aldosterone synthase.

10. The method of claim 7, wherein the disorder or the disease is selected from hypokalemia, hypertension, congestive heart failure, renal failure, restenosis, atherosclerosis, syndrome X, obesity, nephropathy, post-myocardial infarction, coronary heart diseases, increased formation of collagen, or fibrosis and remodeling following hypertension and endothelial dysfunction.

11. A pharmaceutical composition, comprising:
a therapeutically effective amount of a the compound of claim 1 and one or more pharmaceutically acceptable carriers.

12. A pharmaceutical composition, comprising:
a therapeutically effective amount of the compound according to claim 1 and one or more therapeutically active agents selected from
(i) HMG-Co-A reductase inhibitor or a pharmaceutically acceptable salt thereof;
(ii) angiotensin II receptor antagonist or a pharmaceutically acceptable salt thereof;
(iii) angiotensin converting enzyme (ACE) Inhibitor or a pharmaceutically acceptable salt thereof;
(iv) calcium channel blocker (CCB) or a pharmaceutically acceptable salt thereof;
(v) dual angiotensin converting enzyme/neutral endopeptidase (ACE/NEP) inhibitor or a pharmaceutically acceptable salt thereof;
(vi) endothelin antagonist or a pharmaceutically acceptable salt thereof;
(vii) renin inhibitor or a pharmaceutically acceptable salt thereof;
(viii) diuretic or a pharmaceutically acceptable salt thereof;
(ix) an ApoA-I mimic;
(x) an anti-diabetic agent;
(xi) an obesity-reducing agent;
(xii) an aldosterone receptor blocker;
(xiii) an endothelin receptor blocker; an or
(xiv) CETP inhibitor.

13-23. (canceled)
